SEQUENCE LISTING (40 sequences)

SEQ ID NO: 1 - DNA, 24 bp, Artificial
  Actin_primer(F)
catgtttgagaccttcaacacccc

SEQ ID NO: 2 - DNA, 23 bp, Artificial
  Actin_primer(R)
gccatctcctgctcgaagtctag

SEQ ID NO: 3 - DNA, 18 bp, Artificial
  TIM-3_primer(F
aggtcactccagctcaga

SEQ ID NO: 4 - DNA, 19 bp, Artificial
  TIM-3_primer(R)
ggctgttgacgtagcagta

SEQ ID NO: 5 - DNA, 20 bp, Artificial
  TIM-3_e2_primer(F)
ccctgcagttacactctacc

SEQ ID NO: 6 - DNA, 20 bp, Artificial
  TIM-3_e2_primer(R)
gtatcctgcagcagtaggtc

SEQ ID NO: 7 - DNA, 18 bp, Artificial
  TIM-3_e3_primer(F)
aggtcactccagctcaga

SEQ ID NO: 8 - DNA, 19 bp, Artificial
  TIM-3_e3_primer(R)
ggctgttgacgtagcagta

SEQ ID NO: 9 - DNA, 20 bp, Artificial
  PD-L1_primer(F)
agtgcagattccctgtagaa

SEQ ID NO: 10 - DNA, 20 bp, Artificial
  PD-L1_primer(R)
tgggatatcttgttgaggtc

SEQ ID NO: 11 - DNA, 21 bp, Artificial
  TNF-alpha_primer(F)
atgagcacagaaagcatgatc

SEQ ID NO: 12 - DNA, 21 bp, Artificial
  TNF-alpha_primer(R)
tacaggcttgtcactcgaatt

SEQ ID NO: 13 - DNA, 20 bp, Artificial
  iNOS_primer(F)
tcactgggacagcacagaat

SEQ ID NO: 14 - DNA, 20 bp, Artificial
  iNOS_primer(R)
tgtgtctgcagatgtgctga

SEQ ID NO: 15 - DNA, 23 bp, Artificial
  Bid_primer(F)
agtcaggaagaaatcatccacaa

SEQ ID NO: 16 - DNA, 23 bp, Artificial
  Bid_primer(R)
ctcctcagtccatctcgtttcta

SEQ ID NO: 17 - DNA, 24 bp, Artificial
  Survivin_primer(F)
tcgccaccttcaagaactggccct

SEQ ID NO: 18 - DNA, 24 bp, Artificial
  Survivin_primer(R)
gtttcaagaattcactgacggtta

SEQ ID NO: 19 - DNA, 22 bp, Artificial
  IL-12p35_primer(F)
gacttgaagatgtaccagacag

SEQ ID NO: 20 - DNA, 20 bp, Artificial
  IL-12p35_primer(R)
gagctgagatgtgatgggag

SEQ ID NO: 21 - DNA, 19 bp, Artificial
  IL-12p40_primer(F)
agaggaggggtgtaaccag

SEQ ID NO: 22 - DNA, 19 bp, Artificial
  IL-12p40_primer(R)
gggaacacatgcccacttg

SEQ ID NO: 23 - DNA, 20 bp, Artificial
  IL-23p19_primer(F)
aataatgtgccccgtatcca

SEQ ID NO: 24 - DNA, 20 bp, Artificial
  IL-23p19_primer(R)
catggggctatcagggagta

SEQ ID NO: 25 - DNA, 17 bp, Artificial
  GAPDH_primer(F)
cgtggagtctactggtg

SEQ ID NO: 26 - DNA, 18 bp, Artificial
  GAPDH_primer(R)
ggttcacacccatcacaa

SEQ ID NO: 27 - DNA, 18 bp, Artificial
  TIM-3_primer(F)
aggtcactccagctcaga

SEQ ID NO: 28 - DNA, 19 bp, Artificial
  TIM-3_primer(R)
ggctgttgacgtagcagta

SEQ ID NO: 29 - DNA, 20 bp, Artificial
  PD-L1_primer(F)
agtgcagattccctgtagaa

SEQ ID NO: 30 - DNA, 20 bp, Artificial
  PD-L1_primer(R)
tgggatatcttgttgaggtc

SEQ ID NO: 31 - DNA, 21 bp, Artificial
  TNF-alpha_primer(F)
ccaccacgctcttctgtctac

SEQ ID NO: 32 - DNA, 20 bp, Artificial
  TNF-alpha_primer(R)
agggtctgggccatagaact

SEQ ID NO: 33 - DNA, 20 bp, Artificial
  iNOS_primer(F)
tcactgggacagcacagaat

SEQ ID NO: 34 - DNA, 20 bp, Artificial
  iNOS_primer(R)
tgtgtctgcagatgtgctga

SEQ ID NO: 35 - DNA, 21 bp, Artificial
  IL-12p35_primer(F
gaggacttgaagatgtaccag

SEQ ID NO: 36 - DNA, 21 bp, Artificial
  IL-12p35_primer(R)
ttctatctgtgtgaggagggc

SEQ ID NO: 37 - DNA, 23 bp, Artificial
  IL-12p40_primer(F)
cttgcagatgaagcctttgaaga

SEQ ID NO: 38 - DNA, 22 bp, Artificial
  IL-12p40_primer(R)
ggaacgcacctttctggttaca

SEQ ID NO: 39 - DNA, 20 bp, Artificial
  IL-23p19_primer(F)
cctactaggactcagccaac

SEQ ID NO: 40 - DNA, 20 bp, Artificial
  IL-23p19_primer(R)
ttaagctgttggcactaagg

(19) United States
(12) Patent Application Publication
(10) Pub. No.: US 2023/0213523 A1
(43) Pub. Date: Jul. 6, 2023

(54) METHOD FOR PROVIDING INFORMATION FOR DIAGNOSING OR PREDICTING PROGNOSIS OF CANCER BY MEASURING EXPRESSION LEVEL OF TIM-3 IN CD11B+ CELLS

(71) Applicant: NATIONAL CANCER CENTER, Goyang-si (KR)

(72) Inventor: Eun Jung PARK, Seoul (KR)

(73) Assignee: NATIONAL CANCER CENTER, Goyang-si (KR)

(21) Appl. No.: 17/904,926

(22) PCT Filed: Feb. 25, 2021
(86) PCT No.: PCT/KR2021/002366

§ 371 Date: Aug. 24, 2022

(30) Foreign Application Priority Data
Feb. 25, 2020 (KR) 10-2020-0023273

(51) Int. Cl. G01N 33/574; C12N 5/0784; C12N 5/0786

(52) U.S. Cl. CPC G01N 33/57484; C12N 5/0639; C12N 5/0645; A61K 2039/5154

(57) ABSTRACT

The present invention relates to a method for providing information for cancer diagnosis or prognosis prediction by measuring the expression level of TIM-3 in CD11b+ cells.

In addition, the present invention relates to a composition for cancer diagnosis or prognosis prediction comprising an agent for measuring the expression level of TIM-3 in CD11b+ cells or uses thereof.

Since glial TIM-3 responds positively and uniquely to brain tumors and has specific intracellular and intercellular immune modulatory roles that may differ from TIM-3 in brain tumor microenvironment T cells, it can be effectively used in a composition or a method for providing information for cancer diagnosis or prognosis prediction.

### TECHNICAL FIELD

The present invention relates to a method for providing information for diagnosis of cancer or prediction of cancer prognosis by measuring the expression level of TIM-3 in CD11b+ cells.

In addition, the present invention relates to a composition for diagnosis of cancer or prediction of cancer prognosis comprising an agent for measuring the expression level of TIM-3 in CD1 1b+ cells composition or use thereof.

### BACKGROUND ART

T cell immunoglobulin and mucin domain-3 (TIM-3) is a transmembrane glycoprotein composed of an immunoglobulin variable domain, a mucin domain, and a cytoplasmic tail with a tyrosine phosphorylation motif. Initially, TIM-3 was proposed as a T helper type 1 (Th1)-specific protein selectively expressed in terminally differentiated Th1 cells, but as a result of further studies, TIM-3 has been shown to be expressed on Th17 cells, regulatory T (Treg) cells, and even non-T cells such as dendritic cells (DC), natural killer (NK) cells, monocytes and macrophages. This suggests that TIM-3 has various immune functions depending on specific cell types and cell states. In particular, TIM-3 seems to play both positive and negative roles depending on the situation. For example, TIM-3 can attenuate the response of CD4+ and CD8+ T cells and inhibit T cell activation to induce peripheral tolerance. However, in certain cases TIM-3 may contribute to the elimination of pathological stimuli by participating in the activation of various innate immune cells, including quiescent macrophages. Recent studies have shown that TIM-3 promotes short term effector T cells during viral infection. It has also been reported that dysregulation of TIM-3 expression responded excessively or inappropriately in immune cells.

Experimental and clinical studies have implicated TIM-3 in many diseases, including autoimmune diseases, chronic infections and ischemia. Alterations and dysregulation of TIM-3 expression have been associated with the onset and severity of pathological conditions in patients with multiple sclerosis (MS) and patients infected with human immunodeficiency virus (HIV) or hepatitis virus. In particular, alterations and dysregulation of TIM-3 expression have been associated with high levels of expression of IFN-γ and TNF-α in MS patients and with distortion of Th1-induced Th2 responses in allergic diseases. Moreover, blockade of TIM-3 has been shown to affect the pathological severity of experimental allergic encephalomyelitis (EAE) and the pathogenesis of diabetes in obese diabetic (NOD) mice. It has been shown that blockade of TIM-3 signaling restored proliferation and increased cytokine production in HIV-specific T cells. In these TIM-3-related diseases, negative regulation of Th1 or Th17 immunity by TIM-3 is likely to lead to pathological conditions such as T cell dysfunction or depletion of T cells, distortion of Th2 responses to Th1 signaling, and pro-inflammatory states of Th1 responses. However, little is known about the detailed characterization and role of TIM-3 in certain immune states and distinct diseases, particularly in innate immune-related pathologies.

TIM-3 is also receiving attention as a possible target for immune modulation of cancer. In recent years, much research has been done on immune checkpoint molecules and immune surveillance in the context of cancer growth and eradication, which has led to the development of numerous therapeutic strategies, including therapeutic antibodies targeting immune checkpoint molecules. Studies have suggested that TIM-3 expression was associated with several cancers and that TIM-3 played a role in regulating tumor growth. For example, TIM-3 was highly expressed on CD4+ and CD8+ tumor-infiltrating T cells from patients with non-small cell lung cancer, and TIM-3 expression on CD4+ T cells was associated with poor clinicopathological parameters such as nodular metastasis and advanced cancer. It was found to be characteristically expressed in tumor cells in patients with renal and hepatocellular carcinoma.

Korean Patent Application Publication No. 10-2019-0059304 relates to a method of treating elevated TIM-3, and provides a method of treating a subject with increased TIM-3 expression level due to cancer such as brain cancer.

However, there has been no study or description of whether TIM-3 can affect tumor immunity by being involved in immune surveillance against brain tumors.

### DISCLOSURE

### Technical Problem

The present inventors made diligent efforts to demonstrate the role of TIM-3 in mediating myeloid cell responses in brain tumors, thereby facilitating the development of strategies for timely combination with immune checkpoint molecules or TIM-3-based therapeutics for brain tumors. As a result, it was confirmed that TIM-3 of other cells, such as glial, other than brain tumor microenvironment T cells, can specifically respond to brain tumors and play a role in specific intracellular and intercellular immune regulation, and completed the present invention.

Accordingly, one of objects of the present invention is to provide a method for providing information for diagnosis of cancer or prediction of cancer prognosis by measuring the expression level of TIM-3 in CD11b+ cells.

Another object of the present invention is to provide a composition for diagnosis of cancer or prediction of cancer prognosis comprising an agent for measuring the expression level of TIM-3 in CD1 1b+ cells composition or use thereof.

### Technical Solution

The present invention can provide a method of providing information for diagnosing cancer or predicting prognosis of cancer, comprising the step of measuring the expression of TIM-3 in CD1 1b+ cells and judging it as cancer or predicting that cancer has recurred when the level is lower than that of a normal control group.

According to a preferred embodiment of the present invention, the CD1 1b+ cells may be glia cells, myeloid cells, or peripheral blood mononuclear cells (PBMC).

According to a preferred embodiment of the present invention, the glial cells may be at least one selected from the group consisting of radioactive glia cells, astrocytes, oligodendrocytes, oligodendrocytes progenitor cells and microglia.

According to a preferred embodiment of the present invention, the myeloid cells may be any one or more selected from the group consisting of leukocytes, mast cells, monocytes, macrophages and dendritic cells.

According to a preferred embodiment of the present invention, the cancer may be a brain tumor.

The present invention also can provide a composition for diagnosing cancer or predicting prognosis of cancer comprising an agent for measuring the expression level of TIM-3 in CD11b+ cells and a kit for diagnosing cancer or predicting prognosis of cancer comprising the composition.

### Advantageous Effects

In the present invention, TIM-3 was expressed in both surrounding cells, including tumor cells, glial and T cells, growing in an orthotopic mouse glioma model. However, unlike that of other immune checkpoint molecules, the expression pattern of TIM-3 was lower in tumor-infiltrating CD11b⁺CD45^{mid} glial cells, CD11b⁺CD45^{high} macrophages or peripheral blood mononuclear cells compared to normal cells, and TIM-3 was higher in tumor-infiltrating CD8+ T cells. In addition, TIM-3 affected the expression of several immune-related molecules, including iNOS, PD-L1 and IFN-γ. Therefore, TIM-3 of the present invention can be effectively used in a composition for diagnosing or predicting cancer or a method of providing information for diagnosing cancer or predicting prognosis of cancer.

### DESCRIPTION OF DRAWINGS

FIG. 1 showed that TIM-3 was expressed in both tumor and immune cells growing in a mouse glioma model. Expression of TIM-3 and EGFP was shown in red and green, respectively, and arrows indicated cells expressing EGFP (red arrow, left), TIM-3 (white arrow, right) and both (orange arrow).

FIG. 2 showed that TIM-3 was expressed in both tumor and immune cells growing in a mouse glioma model. Red arrows indicated TIM-3 expressing Iba-1 positive cells.

FIG. 3a showed that glial TIM-3 expression was differentially regulated when exposed to brain tumors compared to other immune checkpoint molecules. It was confirmed that both mouse primary glial cells and rat primary glial cells showed lower TIM-3 expression levels when exposed to mouse GL26 (GL26CM), rat B35 (B35CM), or human brain tumor cell U373 (U373CM) compared to normal astrocyte medium (ACM).

FIG. 3b showed the quantitative results of RT-PCR, FACS or Western blot analysis shown in FIG. 3a.

FIG. 4a showed that glial TIM-3 expression was differentially regulated when exposed to brain tumors compared to other immune checkpoint molecules. It was confirmed that Mouse primary glial cells showed increased PD-L1 expression levels when exposed to mouse GL26 (GL26CM), rat B35 (B35CM) or human brain tumor cells U373 (U373CM) compared to normal astrocyte medium (ACM), whereas there was no change in 4-1BB and PD-1 expression level.

FIG. 4b showed the quantitative results of RT-PCR, FACS or Western blot analysis shown in FIG. 4a.

FIG. 5a showed that TIM-3 level was relatively low in CD11b+ cells (Ipsilateral) in the tumor-bearing region compared to the cells in the tumor-opposite region (Contralateral). It was confirmed that the expression level of TIM-3 was decreased in CD11b⁺CD45^{mid} microglial cells (A, left) and CD11b⁺CD45^{high} macrophages (A, right), and was increased in CD3⁺CD8⁺ and CD3⁺CD8⁻leukocytes (B).

FIG. 5b showed the quantitative results of RT-PCR, FACS or Western blot analysis shown in FIG. 5a.

FIG. 6a showed that TIM-3 level was relatively low in CD11b+ cells (Ipsilateral) in the tumor-bearing region compared to the cells in the tumor-opposite region (Contralateral). The proportion of TIM-3^{low}CD1 1b+ cells was higher, whereas no significant differences were found in TIM-3 levels in CD4⁺ and CD8⁺ T cells, and the proportion of PD-L1^{high}CD11b⁺ cells was increased in PBMCs of GL26 tumor-bearing mice compared to PBMCs of control mice.

FIG. 6b showed the quantitative results of RT-PCR, FACS or Western blot analysis shown in FIG. 6a.

FIG. 7 showed the effect of TIM-3 signaling defects on the expression of several immune-linked genes in glial cells. When exposed to GL26CM or Pam3, the expression level of IL-12p35, IL-12p40, IL-23p19 or iNOS of WT was increased compared to Tim-3mut, but the difference in TNF-α was insignificant.

FIG. 8a showed the effect of TIM-3 signaling defects on the expression of several immune-linked genes in glia. When exposed to GL26CM, the increase in the expression level of PD-L1 in WT was insignificant compared to Tim-3mut, and when infected with Ad-CMV-Cre, the expression level of TIM-3 or PD-L1 was higher.

FIG. 8b showed the quantitative results of RT-PCR, FACS or Western blot analysis shown in FIG. 8a.

FIG. 9 showed that expression of TLR2 and TIM-3 was cross-regulated in response to brain tumors. When exposed to GL26CM or Pam3CSK4, the expression level of TLR2 in WT was increased compared to Tim-3mut, but the expression level of TLR1 or TLR6 was not affected.

FIG. 10 showed the cell surface expression level of TIM-3 on primary glial cells of WT, TLR2-knockout (KO) and TLR4-KO mice. When exposed to GL26CM or Pam3CSK4, TIM-3 level of WT or TLR4-KO was decreased and the level of TLR-2 was increased, whereas in the case of TLR2-KO, there was no change in TIM-3 or TLR-2 level.

FIG. 11 showed that exposure to GL26CM reduced IFN-y levels in co-cultures containing CD8+ T cells and TIM-3 signal-deficient glia (Tim-3mut glia). In addition, it was confirmed that IFN-γ level was higher in TIM-3 ^{high} glial cells infected with Ad-CMV-Cre compared to control cells infected with Ad-CMV.

FIG. 12 showed that the reduction of glial TIM-3 in brain tumors was not affected by oxygen concentration.

FIG. 13 showed that the reduction of glial TIM-3 in brain tumors was not affected by oxygen concentration. The increase of TIM-3 by GL26CM in CD8+ T cells was similar in 20% O₂ and 1% O₂, but the level of TIM-3 was higher in 1% O₂. E wss a schematic diagram showing the properties of TIM-3 under the brain tumor microenvironment.

FIG. 14 showed the construction of an orthotopic brain tumor mouse model. A showed a schematic of lentiviral vector pLL3.7 EF1α and EGFP-expressing mouse GL26 glioma cells. B and C showed the coordinates within the skull in the right brain hemisphere of mice transplanted with GL26 cells transfected with pLL3.7 EF1α. C showed the results of immunohistochemistry performed using antibodies against TIM-3 (left), IgG (right) and GL26 (EGFP).

FIG. 15 showed the expression pattern of TIM-3 in the brain tumor microenvironment. It was confirmed that TIM-3 was expressed in CD3⁺CD45⁺, F4/80⁺, CD11b⁺, CD11b⁺ CD45^{high} or CD1 1b⁺CD45^{mid} cells.

FIG. 16 showed a unique expression pattern of TIM-3 in primary gliomas (P.Glia or P.Microglia) and gliomas (GL26 or B35).

FIG. 17 showed the expression patterns of PD-L1, 4-1BBL, PD-1 and 4-1BB in primary glial (P.Glia) and glioma (GL26 glioma).

FIGS. 18 to 20 showed the expression phenotype of CD1 1b+ primary glial cultured from C57BL/6 mice. Primary cultured glial cells were distinguished by the indicated markers.

FIG. 21 showed the results of analysis by flow cytometry using the indicated antibodies after culturing primary glial cells with 5% MEM, ACM or GL26CM for 24 hours.

FIG. 22 showed that TIM-3 expression pattern of mice bearing CT-2A murine glioma cells was similar to the GL26 model.

FIG. 23 showed that both TIM-3 signaling defects and TIM-3 overexpression affected the expression of several immune-binding genes such as IL-12p35, IL-12p40, IL-23p19, TNF-α or PD-L1 in glia.

FIG. 24 showed that the effects of both TIM-3 signaling defects and TIM-3 overexpression on the expression of several immune-binding genes, such as iNOS to IL-12p40, IL-23p19 or PD-L1, in glial cells.

FIG. 25 showed the expression level of TIM-3 according to the presence or absence of brain tumor in CD45+CD11+ cells.

FIG. 26 showed the expression level of TIM-3 in CD45+CD11 cells of brain tumor bearing mouse models.

FIG. 27a showed the differential expression levels of TIM-3 in CD1 1b+ cells (zone A or B) or CD1 1b-cells (zone C or D) in peripheral blood mononuclear cells (PBMCs).

FIG. 27b showed the expression level of TIM-3 in CD1 1b+ cells of the mouse tumor group compared to that of the normal control group.

FIG. 28 showed the expression level of TIM-3 according to the CD11b cell expression level in normal control or brain tumor-bearing mouse models.

FIG. 29 showed the ratio according to the expression level of TIM-3 in CD11b+ cells of normal control or brain tumor bearing mouse models.

### MODE OF THE INVENTION

Hereinafter, the present invention will be described in more details.

As described above, TIM-3 is expressed not only by tumor-associated immune cells but also by tumor cells themselves in various tumors, and is known to exhibit various characteristics and roles for each type of carcinoma or environment. Accordingly, the present inventors sought to develop an efficient therapy targeting TIM-3 in specific tumors.

Aggressive brain tumors, such as glioblastoma (GBM), generally have high overall mortality and poor prognosis. Current standard treatment for glioma includes surgical resection, radiation or combination with temozolomide or adjuvant chemotherapy. Clinical trials and experimental studies are currently underway to improve the treatment efficacy, among which immune-based therapy is considered a promising approach. However, brain tumors hide behind the blood-brain barrier, so little is known about their local immune microenvironment. Brain tumors are composed of several types of cells, including tumor cells, infiltrating immune cells, or brain resident immune cells such as microglial cells. In particular, macrophages and microglial cells have been found to constitute up to 34% of immune cells infiltrating brain tumors.

Accordingly, in order to define the precise characterization of TIM-3 in the brain tumor microenvironment, the present inventors first investigated the expression pattern of TIM-3 using tissues and tumor-infiltrating cells in a mouse intracranial brain tumor model established with EGFP-expressing GL26 neurons. (Example 1). Immunohistochemistry and FACS analysis confirmed that TIM-3 was expressed in both glioma cells, T cells and surrounding non-tumor cells including glial cells. In addition, the expression level of TIM-3 in the glial cells of the brain tumor microenvironment exposed to the brain tumor CM (conditioned-media) was significantly reduced (Example 2). On the other hand, under the same conditions, the level of PD-L1 increased, and the level of 4-1BB and PD-1 remained unchanged. According to the in vitro results, TIM-3 levels were relatively lower in tumor-infiltrating CD1 1b⁺CD45 ^{mid} cells in the tumor-bearing hemisphere of the intracranial brain tumor model compared to the non-tumor hemisphere. Conversely, TIM-3 levels in tumor-infiltrating CD8+ T cells were higher than in the contralateral region. These results suggested that glial TIM-3 may have unique features and functions different from that of TIM-3 expressed on T cells in the brain tumor microenvironment.

To determine whether defective glial TIM-3 could affect representative immune responses under brain tumor conditions, TIM-3 signal-deficient mutant mice (Tim-3mut mice) were used (Example 3). As a result, glial cells from Tim-3mut mice showed altered expression of IL-12p35, IL-12p40, IL-23p19, PD-L1 or iNOS compared to normal glial cells. This suggested that TIM-3 could affect PD-L1 expression in brain tumor glial, although the expression levels of TIM-3 and PD-L1 were differently regulated by brain tumors.

Additionally, it was confirmed that TLR2 and TIM-3 in tumor glial had a mutual effect on each other’s expression level (Example 4). Our previous study showed that glial TLR2 responds rapidly to brain tumors by influencing enhanced cell surface expression, enhancement of various immune signaling and tumor growth. In the present invention, induction of TLR2 expression by GL26 or Pam3CSK4 was significantly reduced in glia with TIM-3 signaling defect. This showed that TLR2 was closely related to the expression of IL-12p35, IL-12p40, IL-23p19, PD-L1 and iNOS in innate immune cells including glia. Considering these results, the interrelationship of TLR2 and TIM-3 may be a pathway leading to differences in the expression of these molecules in glia between Tim-3mut and WT mice. On the other hand, TIM-3 expression by GL26CM or Pam3CSK4 was not reduced in TLR-3 deficient glial (Example 5). Although there have been several reports that TIM-3 and TLR4 could interact in disease-related conditions, in our results, unlike TLR2, the relationship between TIM-3 and TLR4-like glial was insignificant. That is, our results showed that TIM-3 and TLR2 could interact closely and influence each other’s expression in glial when first exposed to brain tumor CM or Pam3CSK4, and TIM-3 and TLR4 depended on cell type or state.

Hypoxia is a hallmark of inflammation-related brain diseases such as tumors and cerebral ischemia. The present inventors found that TIM-3 was hypoxic induced in glial cells after ischemia (H/I) and showed that blockade of TIM-3 significantly reduced infarct size and inflammation (such as neutrophil infiltration or edema) compared to IgG-treated control H/I mice. Accordingly, the present inventors investigated whether hypoxia can affect the reduction of glial TIM-3 caused by brain tumors (Example 6). However, no significant difference was found in TIM-3 expression level of LysM-Hif-1α mouse primary glial cells under hypoxic and normoxic conditions. In addition, TIM-3 expression patterns were similar between normal LysM-Cre Glia and HIF-1α-deficient LysM-Hif-1α glial, and TIM-3 enhancement by GL26CM in CD8+ T cells was similar under hypoxia and normoxia. These results suggested that oxygen levels did not mediate brain tumor-dependent alterations of TIM-3 in glial cells and CD8+ T cells exposed to tumor CM. Given these results, there may be factors that regulate differential expression of TIM-3 in glial and CD8+ T cells regardless of oxygen levels.

Tumor progression is regulated by interactions between tumor cells and their surrounding immune cells. Immune cells cooperate to fight and destroy emerging tumor cells, while tumor cells train immune cells to suppress anti-tumor immunity or promote tumor progression. In the present invention, we confirmed that the tumor CM-triggered increase in IFN-γ secretion mediated by the interaction between glial and CD8+ T cells was less pronounced in co-cultures containing Tim-3mut glial and CD8+ T cells compared to in co-cultures containing WT glial and CD8+ T cells. It was found to be less pronounced in co-cultures containing glial and CD8+ T cells. Previously, we reported that glial TLR2 played an essential role in the antigen presentation system by regulating MHCI expression and that the TLR2-MHCI contributed to the proliferation and activation of CD8+ T cells by brain tumors. This suggested that glial TIM-3 interacted with TLR2 to influence the interaction between glial cells and CD8+ T cells in the brain tumor microenvironment.

Overall, in the present invention, considering the function of glial cells, it was confirmed that glial TIM-3 having a unique expression pattern and function can be a target for regulating anti-tumor immunity of the brain, so the present invention can provide immune checkpoint molecules for treating brain tumors.

Accordingly, the present invention provides a method of providing information for diagnosing cancer or predicting prognosis of cancer, comprising the step of measuring the expression of TIM-3 in CD11b+ cells and judging it as cancer or predicting that cancer has recurred when the level is lower than that of a normal control group.

The normal control group may refer to normal CD1 1b+ cells not exposed to the cancer cells.

According to a preferred embodiment of the present invention, the CD1 1b+ cells may be glia cells, myeloid cells, or peripheral blood mononuclear cells (PBMC).

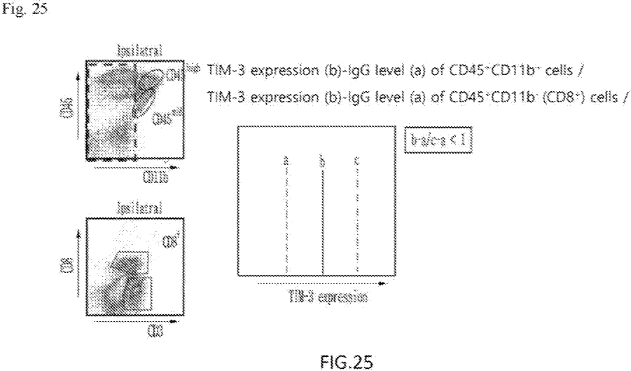

TIM-3 expression level of tumor-infiltrating CD11b+ cells is lower than that of normal control CD11b+ of glia cells or myeloid cells. Specifically, the ratio of the value obtained by subtracting the IgG expression value (a) from the TIM-3 expression value (c) of CD45+CD11b-cells to the value obtained by subtracting the IgG expression value (a) from the TIM-3 expression value (b) of CD45+CD11b+ cells is less than 1 (b-a/c-a < 1). The CD45+CD11b-cells may preferably be CD45+CD11b-CD8+ cells. The CD45+ cells may be CD45 ^{mid} cells or CD45 ^{high} cells (FIG. 25).

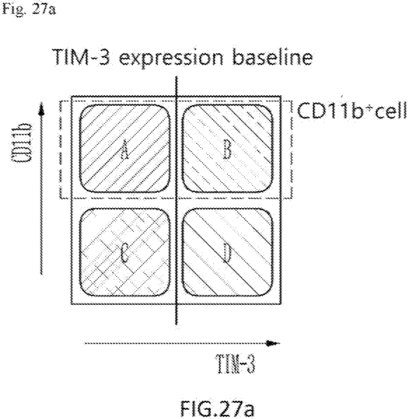

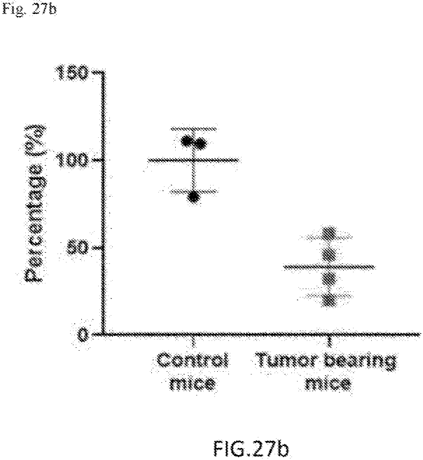

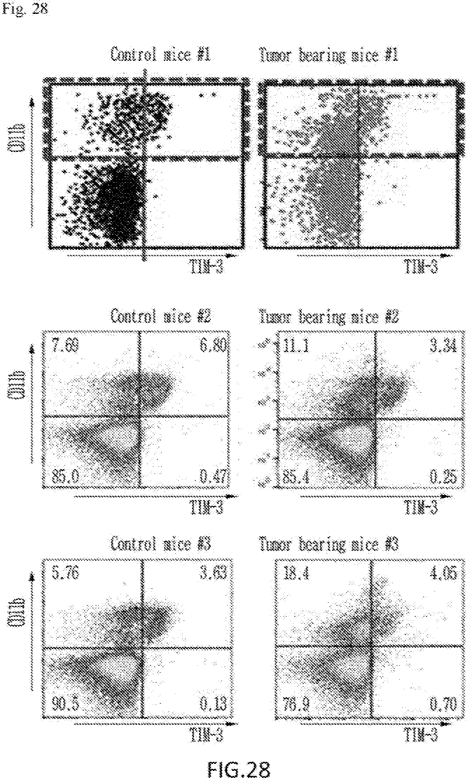

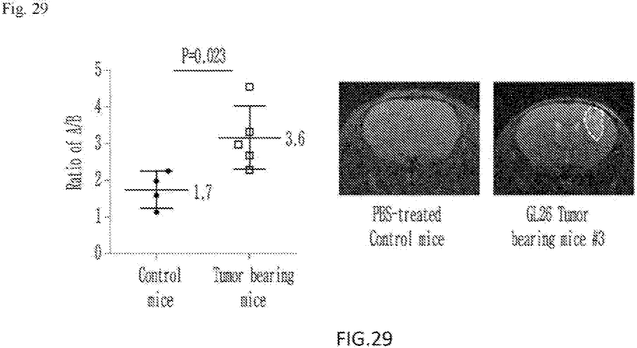

When the peripheral blood mononuclear cells (PBMC) of the normal control group and the tumor-infiltrating group were isolated and comparatively analyzed, the TIM-3 expression level of CD11b+ cells of the tumor-infiltrating PBMC was significantly higher than the TIM-3 expression level of the CD11b+ cells of the normal control PBMC. Specifically, the expression level of TIM-3 in CD1 1b-cells isolated from PBMC mostly corresponds to region C of FIGS. 27. Based on the maximum TIM-3 expression in the C region (TIM-3 expression baseline), in the tumor-infiltrating group, the ratio of the number of cells in region B (CD1 1b+ cells with a TIM-3 expression level higher than the maximum TIM-3 expression in C zone) to the number of cells in region A (CD1 1b+ cells with a lower TIM-3 expression level than TIM-3 expression maximum in C region) among CD1 1b+ cells was measured to be 2.5 or more, preferably 3 or more. The average ratio of the tumor-infiltrating group was 3.6, and the average ratio in the normal control group was 1.7. That is, it could be seen that the expression level of TIM-3 was remarkably low in the tumor-infiltrating group.

That is, through the above results, the expression level (Mean Value) of TIM-3 in CD11b+ of the tumor-infiltrating group was confirmed to be about 40% compared to the expression level of the normal control group.

According to a preferred embodiment of the present invention, the glial cells may be any one or more selected from the group consisting of radioactive glial cells, astrocytes, oligodendrocytes It may be any one or more selected from the group consisting of glial cells (oligodendrocyte), oligodendrocyte progenitor cells and microglia.

According to a preferred embodiment of the present invention, the myeloid cells may be any one or more selected from the group consisting of leukocytes, mast cells, monocytes, macrophages and dendritic cells.

According to a preferred embodiment of the present invention, the cancer may be a brain tumor. The brain tumor may be any one or more selected from the group consisting of anaplastic astrocytomas, glioblastomas, meningiomas, pituitary tumors, schwannomas, CNS lymphoma, oligodendrogliomas, ependymomas, low-grade astrocytomas, medulloblastomas, astrocytic tumors, Pilocytic astrocytoma, diffuse astrocytomas, pleomorphic xanthoastrocytomas, subependymal giant cell astrocytomas, anaplastic oligodendrogliomas, oligoastrocytomas, anaplastic oligoastrocytomas, myxopapillary ependymomas, subependymomas, ependymomas, anaplastic ependymomas, astroblastomas, chordoid gliomas of the third ventricle, gliomatosis cerebris, glangliocytomas, desmoplastic infantile astrocytomas, desmoplastic infantile gangliogliomas, central neurocytomas, cerebellar liponeurocytomas, paragangliomas, ependymoblastomas, supratentorial primitive neuroectodermal tumors, choroids plexus papilloma, pineocytomas, pineoblastomas, moderately differentiated pineal parenchymal tumors of intermediate differentiation, hemangiopericytomas, tumors of the sellar region, craniopharyngioma, capillary hemangioblastoma and Primary CNS lymphoma.

In addition, the present invention can provide a composition for diagnosing cancer or predicting prognosis of cancer comprising an agent for measuring the expression level of TIM-3 in CD1 1b+ cells and a kit for diagnosing cancer or predicting prognosis of cancer.

Since the CD11b+ cells and cancer are the same as those targeted in the method for providing information for diagnosing cancer or predicting prognosis of cancer, the description thereof is replaced by the above description.

The kit of the present invention may consist of one or more other component compositions, solutions, or devices suitable for commonly used expression level analysis methods. For example, a kit for measuring protein expression level may include a substrate, a suitable buffer, a secondary antibody labeled with a chromogenic enzyme or a fluorescent substance, a chromogenic substrate, and the like for immunological detection of the antibody.

The kit of the present invention may include a sample extraction means for obtaining a sample from the subject to be evaluated. The sample extraction means may include a needle or syringe or the like. The kit may include a sample collection container for receiving the extracted sample, which may be liquid, gaseous or semi-solid. The kit may further include instructions for use. Measurement of TIM-3 in the sample can be performed on whole samples or processed samples.

Hereinafter, the present invention will be described in more details through examples. These examples are only for illustrating the present invention, and it is obvious to those of ordinary skill in the art that the scope of the present invention is not to be interpreted as being limited by these examples.

### Materials and Methods

### Animal

Sprague-Dawley (SD) rats and C57BL/6 were purchased from ORIENT BIO. B6.129-Tlr2tm1Kir/J, B6(Cg)-Tlr4tm1.2Karp /J and B6N.129S Havcr2tm1Bmed /J (Tim-3mut) were purchased from Jackson Laboratory. Mice carrying the HIF-1α-phlox allele (HIF-1 a^{+f/+f}) were obtained from Dr. Randall Johnson, and mice lacking HIF-1α in bone marrow lineage cells were were obtained by crossing HIF-1α^{+f/+f}mice with LysM-Cre transgenic mice. A mouse (FSF-TIM-3) with a gene targeting the Rosa26 position in the Flag-TIM-3 structure was prepared by Dr. Lawrence P. Cain. All animals were maintained and housed under SPF conditions in an AAALAC accredited National Cancer Center animal facility. All animal procedures were performed in accordance with the ARRIVE guidelines and NCC guidelines for the care and use of laboratory animals. This protocol was approved by the NCC Animal Experimental Ethics Committee (grant no.: NCC-11-125). To avoid bias, the animal studies of the present invention were appropriately randomized in a blinded fashion with respect to genotype and treatment.

### Cell Culture

Murine CT-2A glioma Cell line, rat B35 cell line and human U373MG cell line were purchased from MERCK (# SCC194), ATCC (# CRL-2754) and Sigma (# 89081403), respectively. The murine GL26 cell line was provided by Dr. Yonggi Hong (Catholic University, Korea). For EGFP-expressing GL26 cells, GL26 cells were transfected with PLL3.7.EF1 α producing lentivirus LV-EGFP, and cells were sorted by gating to 90% purity using FACSAria (BD Bioscience). All cell lines used in the experiment were between passages 2 and 15, were regularly confirmed to be free of mycoplasma contamination (the most recent date was December 2019) and were certified by STR analysis (NCC Genomic Core).

### Primary Glial Culture

Primary glial cells were cultured from the brain cortex of 1 to 3 day-old SD rats and mice as follows: the cortex was triturated into single cells in MEM (Sigma) containing 10% FBS (Hyclone) and 75 cm²T-Flasks (hemisphere/flask for mice) were plated and incubated for 2 weeks. The proportion of microglia in mixed glial cultures was demonstrated to be 30-50% by FACS analysis using anti-CD11b antibody. Microglial cells were detached from the flask by gentle shaking and applied to a nylon mesh to remove astrocytes and clumps of cells. Cells were plated in 24-well plates (5×10⁴ cells/ well). After removing the microglia, primary astrocytes were prepared by trypsinization. The cells were demonstrated to be >95% true microglial cells and astrocytes due to their characteristic morphology and the presence of the microglial cell marker CD11b or the astrocyte marker GFAP. Conditioned media from tumor cells and normal astrocytes were prepared by culturing the tumor cells and primary astrocytes for 48 hours. The medium was collected from the culture dish, centrifuged at 1500 rpm for 10 minutes and then filtered through a 0.2 µm syringe filter (Pall Corporation). 100% conditioned medium was used in this study.

### Intracranial Brain Tumor Model

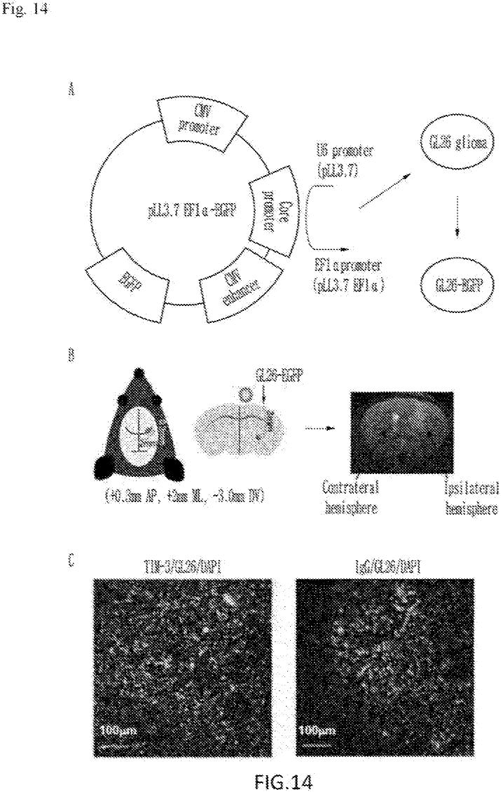

GL26 cells or CT-2A glioma cells were implanted in the right cerebral hemisphere of 8-10 week old female B6 mice. To generate a mouse brain tumor model, 3 × 10⁵ tumor cells were sterically implanted into the brain using the following coordinates: AP = +0.3 mm; ML = +2 mm; and DV =-3 mm in bregma (FIG. 14 B).

### Isolation of Tumor-Infiltrating Immune Cells

Mouse tumor-infiltrating immune cells were isolated using the discontinuous Percoll gradient method. Specifically, animals were anesthetized and cardiovascularized with ice-cold PBS. The brain was removed and sliced 1 mm thick using a stainless steel brain matrix. A brain slice section (3 mm) containing the injection site was divided into left and right hemispheres. Left and right hemispheres were enzymatically digested with DNase I (100 U/ml) and collagenase (0.1 mg/ml) in serum-free DMEM (high clone) at 37° C. for 15 minutes. After quenching the digestion by adding complete medium, the tissue was forced through a 70 µm mesh to obtain a cell suspension and separated with a discontinuous 35%/70% Percoll gradient, and lymphocytes were collected from the interface. The collected fractions were washed and centrifuged to remove Percoll to obtain microglial cells. T cells were further differentiated by flow cytometry using fluorochrome-binding antibodies to CD45, CD3, CD4, CD8 and CD11b (for mice).

### TIM-3 Promoter Assay

A 1517-bp fragment of the mouse TIM-3 promoter (1517 to +1 for start codon) was PCR amplified from genomic DNA and cloned into PGL3 basic vector (Promega). Mouse primary mixed glial cells were transfected using Lipofectamine 2000 (Invitrogen) according to the manufacturer’s instructions. After transfection, cells were incubated with B35CM or ACM for 6 hours, and reporter gene activity was measured with a luciferase assay system (Promega). β-galactosidase activity was measured against normalization of transfection efficiency.

### Flow Cytometry

Flow cytometry was performed using the following antibodies, then acquired with either FACSCalibur or FACSVerse system (BD): PerCP-Cy5.5-binding anti-mouse CD11b (eBioscience, # 45-0112, 0.05 µg ml⁻¹), anti-mouse CD45 (BD Bioscience, #563891, 0.05 µg ml⁻¹; eBioscience, # 17-0451, 0.01 µg ml⁻¹), anti-mouse F4/80 (eBioscience, # 45-4801, 0.02 µg ml⁻¹), anti-mouse TIM-3 (eBioscience, # 12-5870, 0.5 µg ml⁻¹), anti-mouse CD3 (eBioscience, # 11-0031, 0.05 µg ml⁻¹), anti-mouse CD8 (BD Bioscience, # 551162, 0.01 µg ml⁻1; # 563898, 0.01 µg ml⁻¹). Goat anti-TIM-3 (R&D Systems, AF1529, 2 µg ml⁻¹) and alexa-647-binding secondary antibody (Molecular Probe, # A-21447, 2 µg ml⁻¹) were stained with cells of rat origin. APC-binding TIM-3 (Miltenyi Biotec, # 130-102, 3 µg ml⁻¹), PE-bound PD-1 (BD Bioscience, # 551892, 0.1 µg ml⁻ ¹), PE-bound PD-L1 (BD Bioscience, # 558091, 0.1 µg ml⁻¹), PE-bound 4-1BB (eBioscience, # 12-1371, 0.1 µg ml⁻¹), and PE-bound 41BB-L (eBioscience, # 12-5901, 1 µg ml⁻¹) were used for mouse-derived cells; PE-cyanine 7 binding CD11b (eBioscience, #25-0112), APC-binding CD3 (eBioscience, # 17-0031, 0.05 µg ml⁻¹), APC eFlour780-binding CD4 (eBioscience, # 47-0041, 0.05 µg ml⁻¹), BV421-bound CD8 (BD Bioscience, # 563898, 0.05 µg ml⁻¹), PE-bound TIM-3 (eBioscience, # 12-5870, 0.5 µg ml⁻¹) were used for staining mouse PBMCs.

### Immunostaining

For immunohistochemistry, animals were anesthetized and myocardial perfused with PBS followed by perfusion with 4% (w/v) paraformaldehyde in freshly prepared PBS (pH 7.4). Brains were removed from 30% sucrose, fixed and cryopreserved. OCT-bearing brains were dissected on a freeze sliding microtome. Brain sections were pretreated with 0.1% Triton-X100 and blocked with 5% normal serum of the same host species as the labeled secondary antibody. The sections were incubated with the following primary antibodies: goat anti-TIM3 (R & D Systems, 2 mg ml⁻ ¹) and rabbit anti-IBA1 (Wako, # 019-19741, 2.5 µg ml⁻¹). Samples were then stained with the appropriate Alexa-647 or -546 bound secondary antibody (Molecular Probe), counterstained with Hoechst 33342 and mounted with fluorescence mount medium (Dako). For immunocytochemistry, cells grown on coverslips were fixed in ice-cold methanol and washed twice with PBS. Cells were incubated with antibodies specific for TIM-3 followed by incubation with secondary conjugated Alexa-488. Fluorescence images were obtained using an LSM780 confocal laser scanning microscope and analyzed with Zen software (Carl Zeiss).

### RT-PCR Analysis

Total RNAs were isolated using Easy-Blue (iNtRON) and cDNA was synthesized using avian myeloblastosis virus reverse transcriptase (TaKaRa) according to the manufacturer’s instructions. PCR was performed with the indicated primers. The primers used for PCR were as shown in Table 1 below.

TABLE 1

| Target | | Primer Sequence | SEQ ID NO |
|---|---|---|---|
| Actin | F | 5′-CATGTTTGAGACCTTCAACACCCC-3′ | 1 |
| | R | 5′-GCCATCTCCTGCTCGAAGTCTAG-3′ | 2 |
| TIM-3 | F | 5′-AGGTCACTCCAGCTCAGA-3′ | 3 |
| | R | 5′-GGCTTGTTGACGTAGCAGTA-3′ | 4 |
| TIM-3 _e2 | F | 5′-CCCTGCAGTTACACTCTACC-3′ | 5 |
| | R | 5′-GTATCCTGCAGCAGTAGGTC-3′ | 6 |
| TIM-3 _e3 | F | 5′-AGGTCACTCCAGCTCAGA-3′ | 7 |
| | R | 5′-GGCTTGTTGACGTAGCAGTA-3′ | 8 |
| PD-L1 | F | 5′-AGTGCAGATTCCCTGTAGAA-3′ | 9 |
| | R | 5′-TGGGATATCTTGTTGAGGTC-3′ | 10 |
| TNF-α | F | 5′-ATGAGCACAGAAAGCATGATC-3′ | 11 |
| | R | 5′-TACAGGCTTGTCACTCGAATT-3′ | 12 |
| iNOS | F | 5′-TCACTGGGACAGCACAGAAT-3′ | 13 |
| | R | 5′-TGTGTCTGCAGATGTGCTGA-3′ | 14 |
| Bid | F | 5′-AGTCAGGAAGAAATCATCCACAA-3′ | 15 |
| | R | 5′-CTCCTCAGTCCATCTCGTTTCTA-3′ | 16 |
| Survivin | F | 5′-TCGCCACCTTCAAGAACTGGCCCT-3′ | 17 |
| | R | 5′-GTTTCAAGAATTCACTGACGGTTA-3′ | 18 |
| IL-12p35 | F | 5′-GACTTGAAGATGTACCAGACAG-3′ | 19 |
| | R | 5′-GAGCTGAGATGTGATGGGAG-3′ | 20 |
| IL-12p40 | F | 5′-AGAGGAGGGGTGTAACCAG-3′ | 21 |
| | R | 5′-GGGAACACATGCCCACTTG-3′ | 22 |
| IL-23p19 | F | 5′-AATAATGTGCCCCGTATCCA-3′ | 23 |
| | R | 5′-CATGGGGCTATCAGGGAGTA-3′ | 24 |

### Quantitative Real-time PCR

Quantitative real-time PCR was performed with a Roche LightCycler 480 Real-Time PCR system (Roche Applied Science) using the QuantiFast SYBR Green PCR kit (Qiagen). LigthCycler 480 Quantification Software version 1.5 was used for real-time PCR analysis. The primers used for PCR were as shown in Table 2 below.

TABLE 2

| Target | | Primer Sequence | SEQ ID NO |
|---|---|---|---|
| GAPDH | F | 5′-CGTGGAGTCTACTGGTG-3′ | 25 |
| | R | 5′-GGTTCACACCCATCACAA-3′ | 26 |
| TIM-3 | F | 5′-AGGTCACTCCAGCTCAGA-3′ | 27 |
| | R | 5′-GGCTTGTTGACGTAGCAGTA-3′ | 28 |
| PD-L1 | F | 5′-AGTGCAGATTCCCTGTAGAA-3′ | 29 |
| | R | 5′-TGGGATATCTTGTTGAGGTC-3′ | 30 |
| TNF-α | F | 5′-CCACCACGCTCTTCTGTCTAC-3′ | 31 |
| | R | 5′-AGGGTCTGGGCCATAGAACT-3′ | 32 |
| iNOS | F | 5′-TCACTGGGACAGCACAGAAT-3′ | 33 |
| | R | 5′-TGTGTCTGCAGATGTGCTGA-3′ | 34 |
| IL-12p35 | F | 5′-GAGGACTTGAAGATGTACCAG-3′ | 35 |
| | R | 5′-TTCTATTCTGTGTGAGGAGGGC-3′ | 36 |
| IL-12p40 | F | 5′-CTTGCAGATGAAGCCTTTGAAGA-3′ | 37 |
| | R | 5′-GGAACGCACCTTTCTGGTTACA-3′ | 38 |
| IL-23p19 | F | 5′-CCTACTAGGACTCAGCCAAC-3′ | 39 |
| | R | 5′-TTAAGCTGTTGGCACTAAGG-3′ | 40 |

### Western Blot Analysis

Samples for Western blot analysis were separated by SDS-polyacrylamide gel electrophoresis, transferred to a nitrocellulose membrane, and incubated with the following primary antibody: iNOS (Millipore, # 06-573, 1 µg ml⁻¹), TLR1 (Abcam, # ab37068, 1 µg ml⁻¹), TLR2 (R & D systems, # AF47840, 0.1 µg ml⁻¹), TLR6 (Santa cruz, # sc-30001, 0.4 µg ml⁻¹), Bid (Santa cruz, # sc-11423, 0.4 µg ml⁻¹), Survivin (Cell signaling, # 2808, 0.621 µg ml⁻¹), mouse anti-γ-tubulin (Sigma, T5168, 1: 5,000), peroxy multidrug-conjugated goat anti-rabbit (Bio-Rad, # 170-6515, 1: 5,000), peroxidase-conjugated rabbit anti-goat (Zymed, R-21459, 1: 5,000), peroxidase-conjugated goat anti-Mice (Bio-Rad, # 170-6516, 1: 5,000). All experiments were performed independently at least three times.

### Data Analysis

All data were expressed as mean ± standard deviation of the mean (SD) (n = number of individual samples). All statistical analyzes were performed using GraphPad Prism 5 (GraphPad Software). P<0.05 was considered to indicate a significant difference.

### Example 1 Confirmation of TIM-3 Expression Pattern in Orthotopic Glioma Mice

EGFP-expressing mouse GL26 glioma cells were prepared using PLL.3.7 EF1α lentiviral vector system, and the cells were sterically transplanted into the right cerebral hemisphere of C57BL/6 mice (FIG. 14 A, B). Immunohistochemistry was performed on histological sections of optimal cutting temperature (OCT)-embedded frozen brain tissue samples from intracranial brain tumor models at 21 days after transplantation.

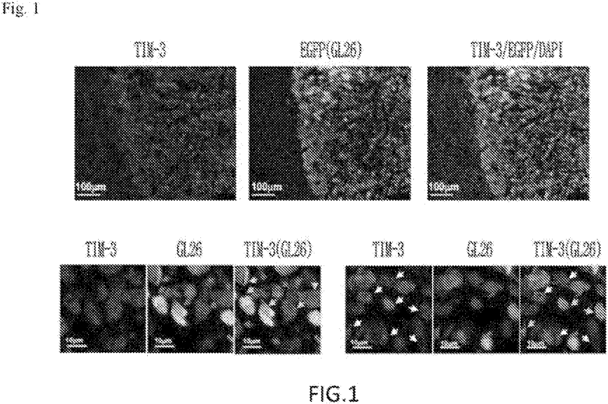

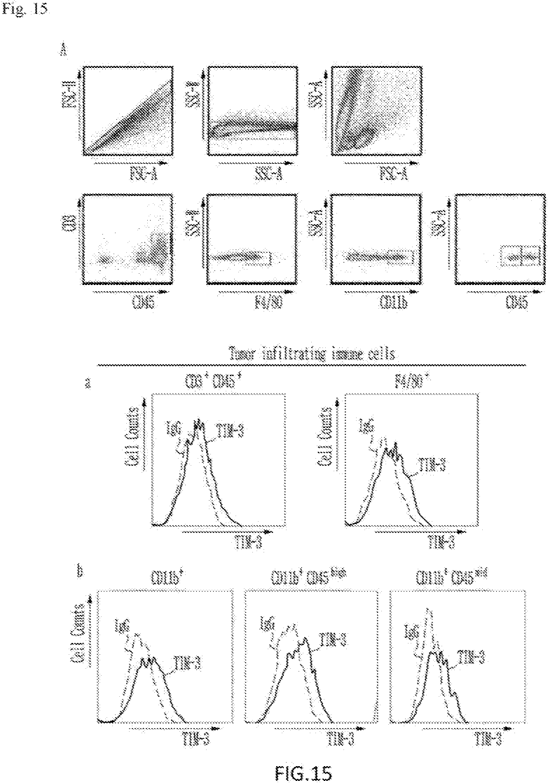

As a result, it was confirmed that both the green fluorescent GL26 glioma cells and surrounding non-tumor cells such as immune cells in the tumor-free region expressed TIM-3 (FIGS. 1 and 14 C). FACS analysis of tumor-infiltrating immune cells isolated on the basis of Percoll gradient from the ipsilateral hemisphere of an intracranial GL26 brain tumor mouse model showed that TIM-3 was expressed not only in CD3+ CD45+ T cells but also in F4/80+ or CD11b+ innate immune cells of brain tumor-bearing hemispheres (FIG. 15).

Glioma is a type of tumor that arises from glial cells that act as brain-resident immune cells. Since TIM-3 expression is detected in CD11b⁺CD45^{mid} microglia, the present inventors compared the basic TIM-3 expression characteristics of CD11b⁺CD45^{mid} glial and GL26 glial in order to analyze the characteristics of TIM-3 in tumor-associated innate immune cells. First, immunohistochemistry was performed using antibodies to ionized calcium binding adapter molecule 1 (Iba1, microglial marker), and healthy B6 mouse primary microglial cells were cultured and then basal levels were investigated using antibodies to TIM-3 as compared to in glioma cell lines. Quantification of fluorescence intensity was performed using Zen software and presented graphically. Each protein level was determined by flow cytometry or immunocytochemistry (e200). Results represent at least 3 independent experiments (n = 3 females in each experiment).

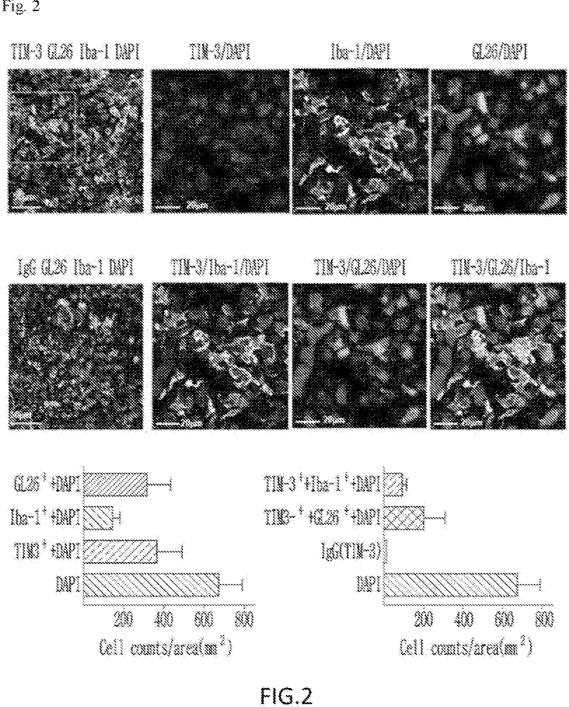

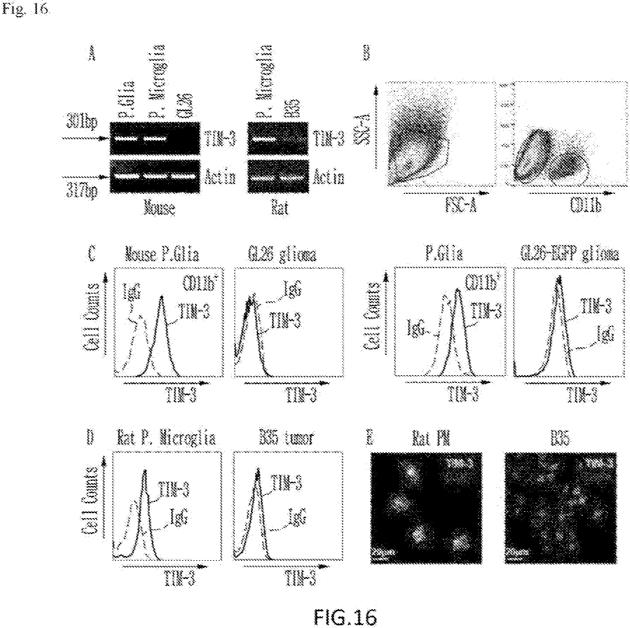

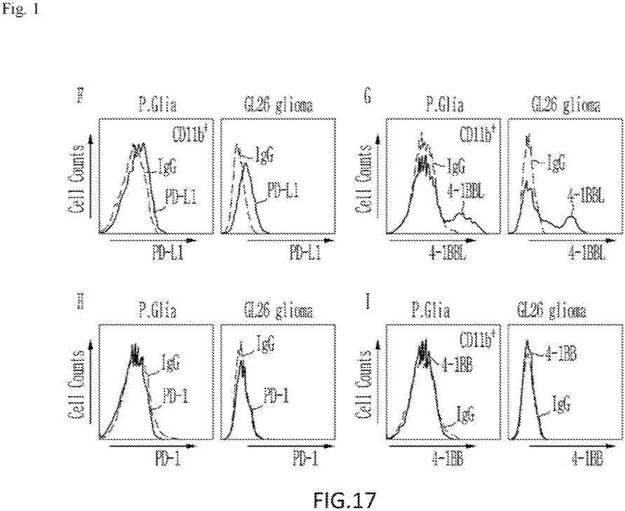

As a result, in the intracranial glioma mouse model, it was confirmed that TIM-3 was expressed on microglial cells in the intracranial glioma mouse model (FIG. 2). In addition, TIM-3 transcripts were expressed at detectable levels in primary culture mixed glia (P. Glia) and microglia cells (P. Microglia) from B6 mice. Basal levels of TIM-3 were significantly lower in GL26 glioma cells than in primary gliomas, both at transcriptional and cell surface levels (FIG. 16 A-C). Similar results were observed in rat primary microglial cells and in B35 rat brain tumor cells (FIG. 16 A, D, E). Unlike TIM-3, PD-L1 was expressed higher in gliomas than in primary gliomas at the cell surface expression level (FIG. 17 F), whereas there was no noticeable difference at the cell surface expression levels of 4-1BBL, PD-1 and 4-1BB (FIG. 17 G-I).

### Example 2 Whether Brain Tumors Affect Glial TIM-3 Expression

### 2-1 in Vitro

Since glial cells are brain resident immune cells that communicate for the first time with pathological changes in abnormal conditions, we wanted to determine whether changes in TIM-3 expression in glial cells occur in response to brain tumors. Mouse glia and rat glia were treated with primary astrocytes (ACM) or media conditioned with indicated brain tumor cells (CM), respectively, and TIM-3 transcript and protein expression levels were determined by RT-PCR and flow cytometry, respectively. Rat primary glia were transfected with TIM-3 luciferase reporter plasmid and stimulated with ACM or B35CM for 6 hours. Relative luciferase activity is expressed as the mean (± SD) of three independent experiments. TIM-3 expression levels were measured by immunocytochemistry (x630) and relative intensity was quantified as image J. Relative expression levels were expressed as mean fold change (± SD).

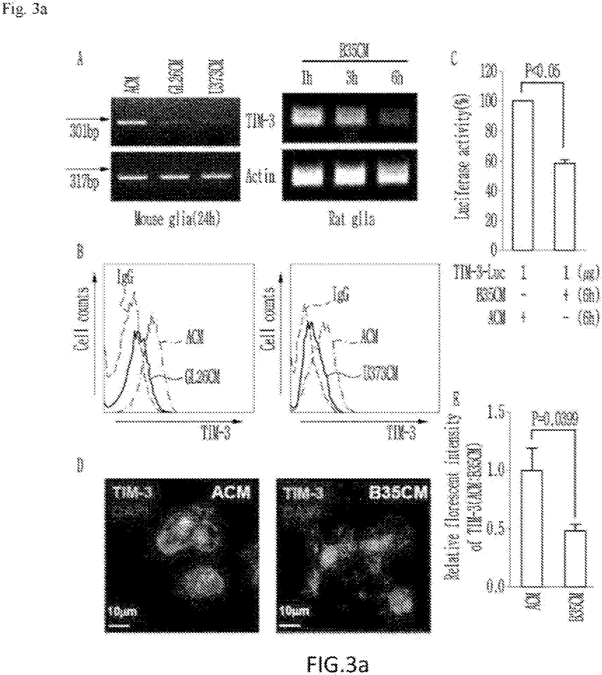

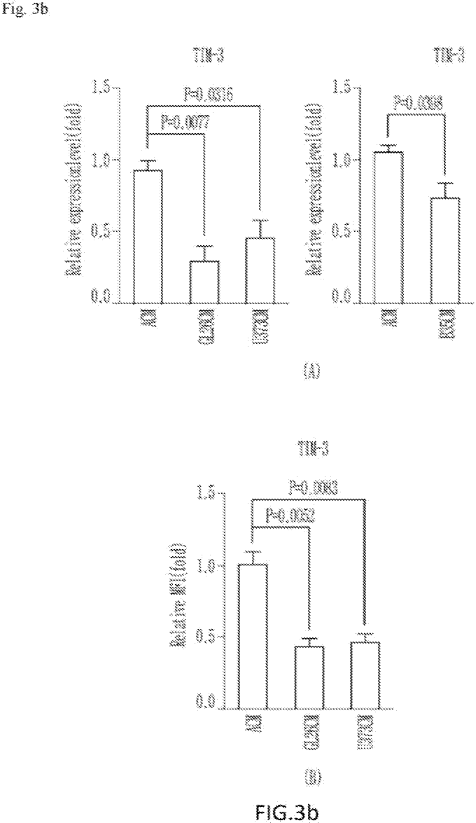

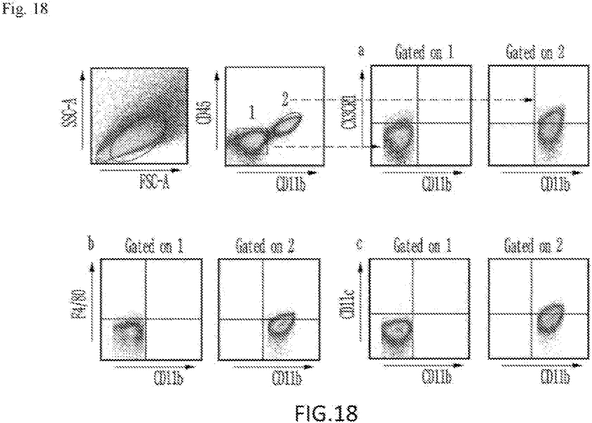

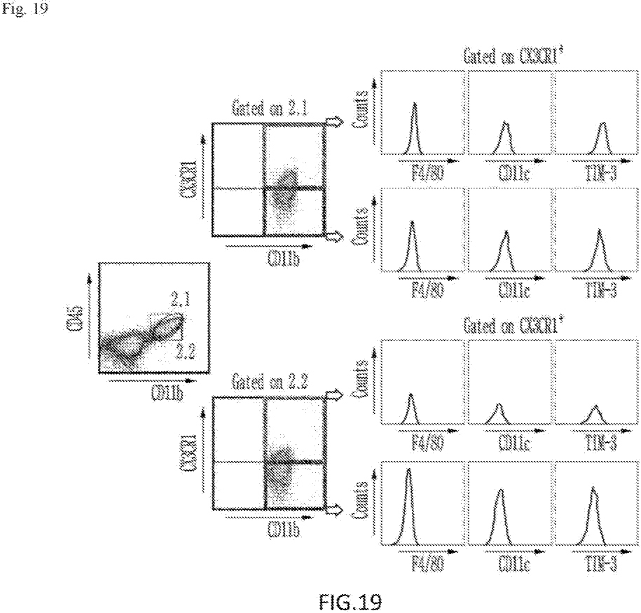

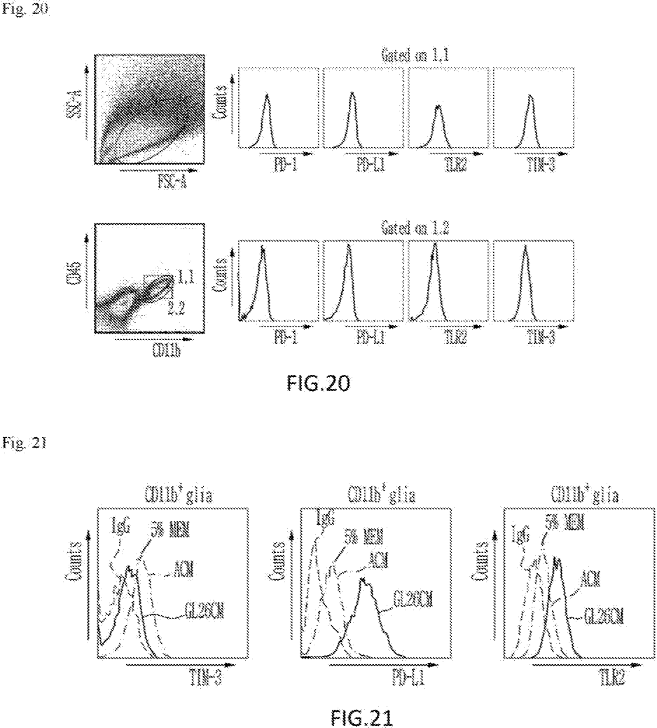

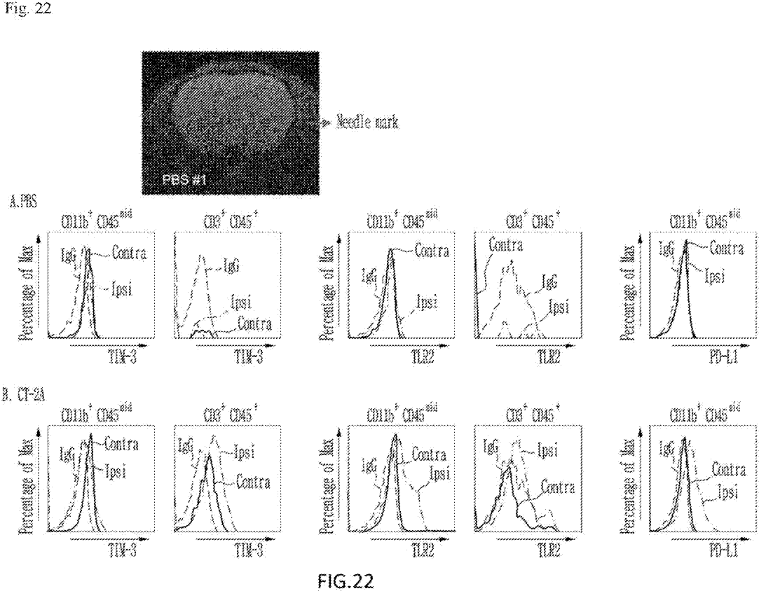

As a result, TIM-3 transcript levels were rapidly and significantly decreased in CM-exposed mouse GL26, rat B35 and human U373 brain tumor cells (FIG. 3a A and FIG. 3b). TIM-3 protein level was also confirmed to be decreased in a time-dependent manner in CD1 1b+ primary glial exposed to brain tumor CM compared with normal astrocyte CM (FIG. 3a B and FIG. 18 A-C). No such alterations were detected when the same cells were exposed to normal primary astrocytes or CM derived from 5% DMEM (FIG. 19 D). In addition, as a result of luciferase analysis, it was found that exposure to B35 tumor cells reduced TIM-3 promoter activity (FIG. 3a C). The results of immunochemical analysis were also similar (FIG. 3a D, E).

To obtain relative information about immune checkpoint molecules and combination therapy approaches, the expression patterns of different immune checkpoint molecules in brain tumor CM-exposed glia were analyzed and compared to the patterns observed for TIM-3. After treatment of mouse primary glia with ACM, GL26CM or U373CM for 24 h, the expression levels of PD-L1, 4-1BB and PD-1 were determined by RT-PCR (PD-L1) or flow cytometry. Results represent at least 3 independent experiments.

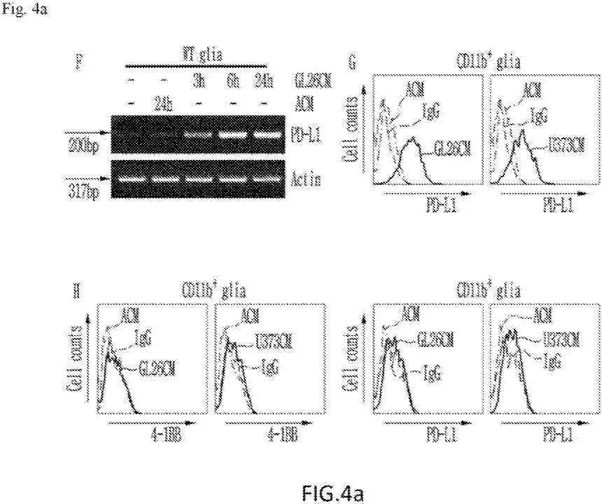

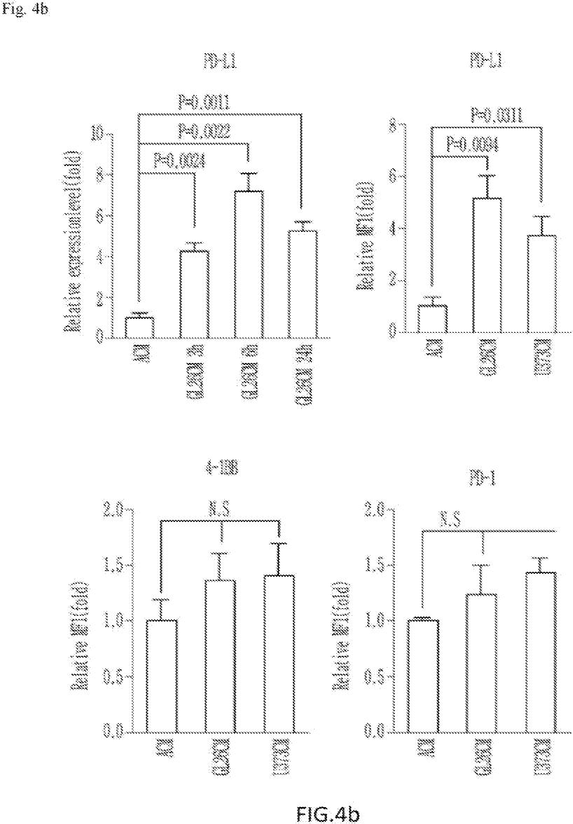

As a result, unlike TIM-3, PD-L1 was significantly upregulated upon exposure to GL26CM and U373CM (FIG. 4a F, G), and the expression levels of 4-1BB and PD-1 did not change (FIG. 4a H, FIG. 4b).

### 2-2 in Vivo

To verify the in vitro results, it was investigated whether the level of glial TIM-3 was reduced by brain tumors in a mouse intracranial brain tumor model. GL26 cells (3×10⁵), CT-2A glioma cells (3 _{×} 10⁵ cells/3 µl PBS) or PBS (3 µl) were transplanted into C57BL/6 mice, and tumor infiltrating cells were isolated from the ipsilateral and contralateral hemispheres of the mice using Percoll gradient centrifugation, stained with anti-CD11b, anti-CD45, anti-CD8 and anti-TIM-3 antibodies, and analyzed by flow cytometry.

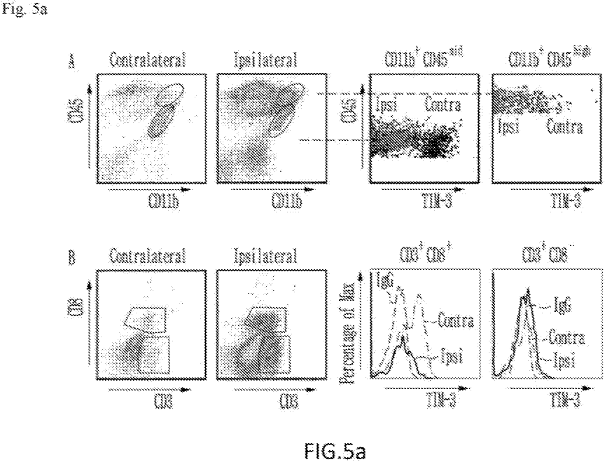

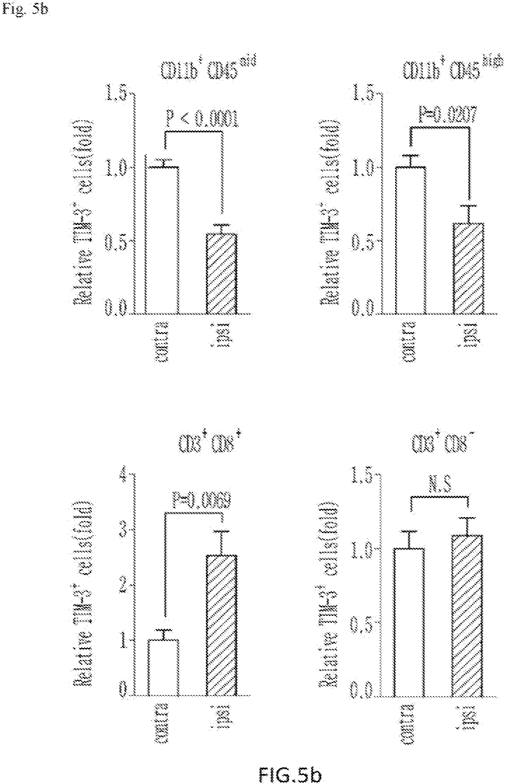

As a result, TIM-3 levels of CD11b⁺CD45^{mid} microglial cells were significantly lower in cells of the ipsilateral tumor bearing region (red) compared to the contralateral region (black), and a similar decrease in TIM-3 levels was observed in sorted CD11b⁺CD45^{high} macrophage cells among tumor infiltrating cells (FIG. 5a A). However, in the same model, TIM-3 levels were significantly increased in tumor-infiltrating CD8+ T cells (FIG. 5a B, FIG. 5b). In addition, the TIM-3 expression pattern of mice bearing CT-2A murine glioma cells was similar to the GL26 model (FIG. 22).

Additionally, we investigated whether TIM-3 levels were different in CD1 1b+ cells sorted among peripheral blood mononuclear cells (PBMCs) from tumor-bearing or healthy control mice. PBMCs were isolated from mice bearing GL26 tumor or control mice using a Histopaque density gradient (HIstopaque-1077, Sigma) and TIM-3 levels in CD1 1b+ cells, CD4+ or CD8+ leukocytes were examined. Results represent at least 3 independent experiments. PD-L1 levels were confirmed in CD11b+ cells.

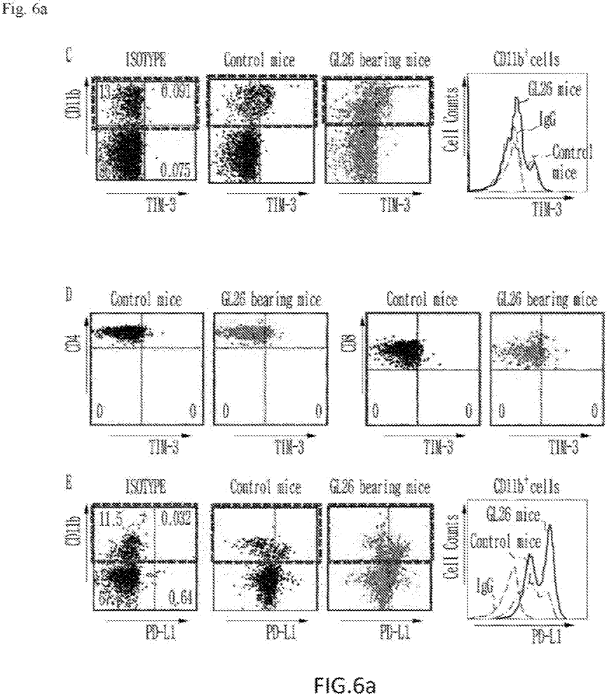

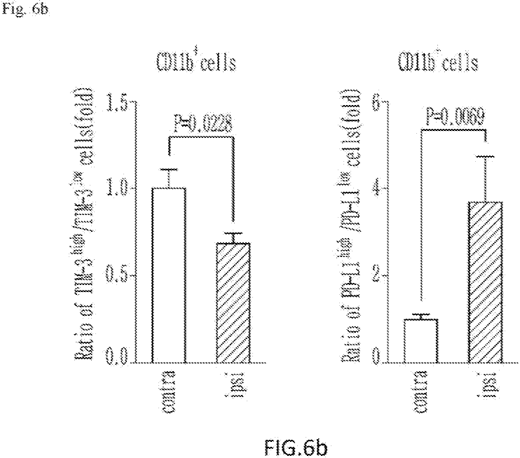

As a result, consistent with the results obtained using tumor-infiltrating cells, it was confirmed that the proportion of TIM-3^{low}CD1 1b+ cells was significantly greater in PBMCs from GL26 tumor-bearing mice compared to control mice (FIG. 6a C). However, no significant difference was observed in TIM-3 levels of CD4 ⁺ and CD8 ⁺ T cell populations obtained from PBMCs of tumor-bearing and control mice (FIG. 6a D), and the ratio of PD-L1^{high} cells was increased in CD11b+ cells from tumor bearing mice versus control mice (FIG. 6a E). Taken together, these in vitro and in vivo data suggested that TIM-3 expression levels in CD11b+ cells, including microglial cells, were down-regulated in response to brain tumors.

### 2-3 Formulating

The results of Example <2-2> were formulated.

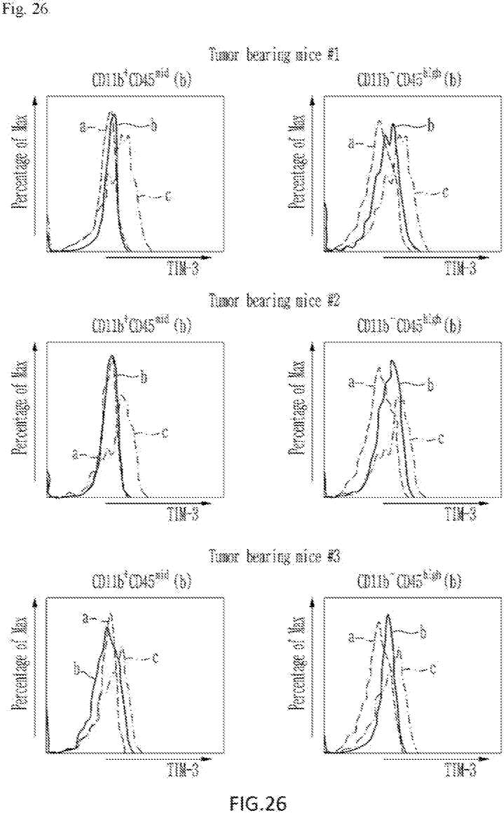

When the level of IgG was a, TIM-3 expression level of CD11b ⁺ CD45 ⁺ cells was b, and TIM-3 expression level of CD1 1b-CD45 ⁺ (CD8⁺) cells was c, it was confirmed that b-a / c-a < 1 (FIG. 25). Table 3 below showed the a, b, and c values of the GL26 tumor-bearing mouse models (FIG. 26). The tumor sizes of mouse models #1, #2 and #3 were 5.301 mm³^{,} 19.989 mm³ and 1.863 mm³, respectively.

TABLE 3

| CD 1b+CD45mid(b) | | CD11b+CD45high(b) | |
|---|---|---|---|
| Mice #1 | | Mice #1 | |
| b | 76.9 | b | 61.8 |
| c | 124 | c | 124 |
| a | 49.3 | a | 31 |
| ba/ca | 0.369 | ba/ca | 0.331 |
| Mice #2 | | Mice #2 | |
| b | 69.7 | b | 80.2 |
| c | 108 | c | 108 |
| a | 49.3 | a | 31 |
| ba/ca | 0.347 | ba/ca | 0.638 |
| b | 52.5 | b | 68.6 |
| c | 84.7 | c | 84.7 |
| a | 49.3 | a | 31 |
| ba/ca | 0.09 | ba/ca | 0.700 |

In addition, as shown in FIG. 27a, when TIM-3 expression maximum value of the C region, which was the CD1 1b-TIM-3 expression level, was determined as the reference (TIM-3 expression baseline), in the tumor-infiltrating group, it was confirmed that the ratio (A/B) of the number of cells in section B (CD11b+ cells with TIM-3 expression higher than the maximum TIM-3 expression in zone C) to the number of cells in section A (CD11b+ cells with TIM-3 expression level lower than the maximum TIM-3 expression in zone C) among CD11b+ cells was 3 or more. The mean of the tumor-infiltrating group was 3.6, and the mean of the normal control group was 1.7.

TIM-3 expression level (Mean Value) in the tumor group CD11b+ cells of the actual mouse was compared numerically by measuring how much it was compared to the expression level of the normal control group. As shown in Table 4 below, it exhibited an expression level (Mean Value) of about 40% compared to the normal control, which was shown in FIG. 27b.

TABLE 4

| | Control mice TIM-3 Geo Mean value | GL26-bearing mice TIM-3 Geo Mean value |
|---|---|---|
| 190429 | 111.238 | 45.87558 |
| | 79.17238 | 58.20773 |
| | 109.5917 | 32.31022 |
| | | 19.97808 |
| Average | 100 | 39.09 |

### Example 3 Effect of TIM-3 Cytoplasmic Domain on Expression of Several Immune-Binding Genes in Glia

The present inventors confirmed the role of glial TIM-3 in the brain tumor microenvironment according to the results of < Example 2>. Specifically, primary microglial cells from Tim-3 mutant (Tim-3mut), harboring a homozygous deletion of TIM-3 cytoplasmic domain and showing intracellular signaling defects, or C57BL/6 wild-type (WT) mice were incubated with GL26CM or normal astrocytes (ACM) for the indicated times, and the, RT-PCR, real-time RT-PCR or Western blot was analyzed. In addition, the cell surface level of PD-L1 was confirmed in primary glia treated with GL26CM or Pam3CSK4 for 24 h. CD1 1b⁺CD45^{mid} Microglial cells were isolated from the contralateral and ipsilateral hemispheres of GL26 tumor-bearing WT and Tim-3mut mice, and PD-L1 levels were examined by flow cytometry. The graph shows the relative levels of PD-L1 from three independent experiments. Primary glia from FSF-TIM-3 mice were treated with Ad-CMV or Ad-CMVCre virus followed by GL26CM for the indicated times. RT-PCR (F) and quantitative real-time PCR (G) analyzes were performed using the indicated primers. Results represent at least 3 independent experiments. Relative expression levels are expressed as mean fold change (± SD).

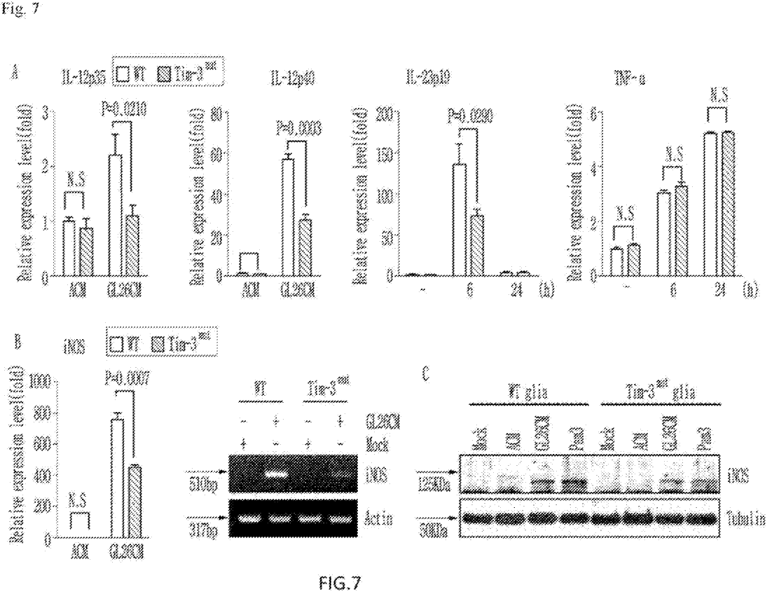

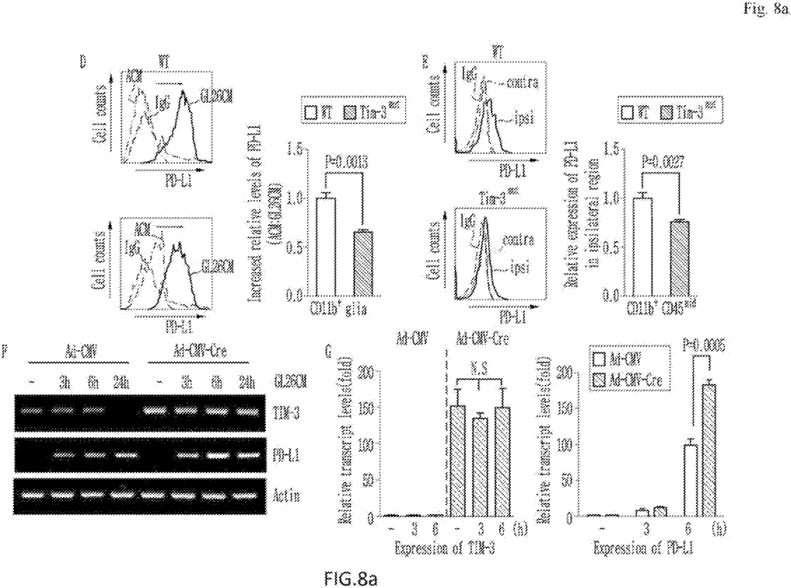

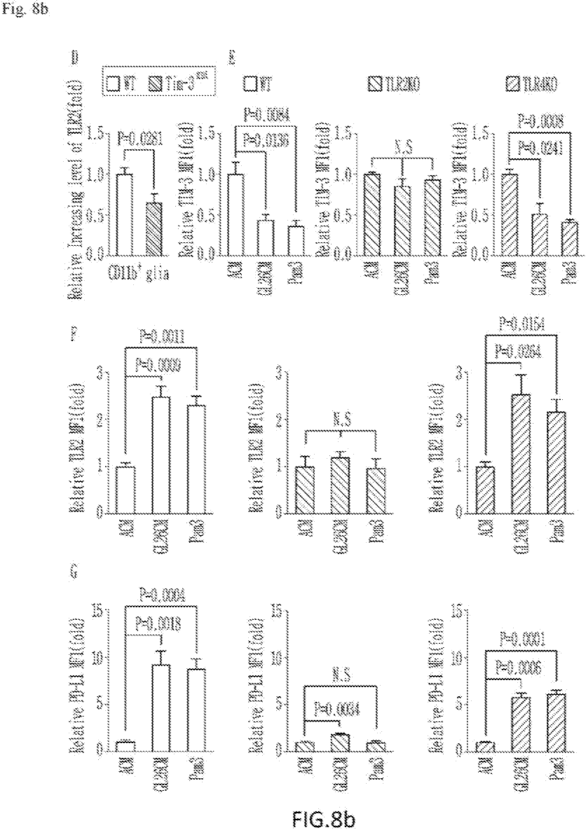

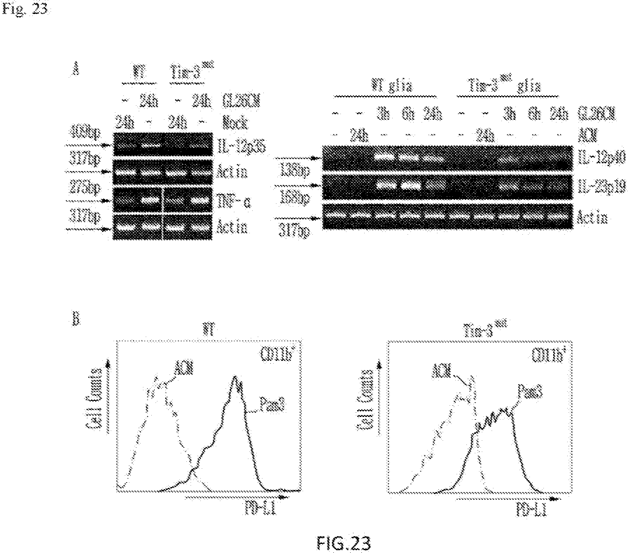

As a result, conventional RT-PCR and real-time RT-PCR showed that several inflammation-related genes, including IL-12p35, IL-12p40 and IL-23p19, were significantly increased in normal WT glial exposed to GL26CM compared to the ACM-treated control group, but decreased in Tim-3mut glia exposed to GL26CM (FIG. 7A and FIG. 23 A). Under the same conditions, some molecules, including TNF-α, showed similar expression levels in WT and Tim-3mut glia (FIG. 7A ). In addition, induction of iNOS expression by GL26CM or Pam3CSK4 was relatively low at RNA and protein levels in Tim-3mut glial compared to WT glial (FIG. 7B, C ). In addition, GL26CM-induced enhancement of PD-L1 was observed to be insignificant in TIM-3 signaling-deficient CD11b+ glial (FIG. 8aD) or Tim-3mut CD11b⁺glial treated with Pam3CSK4 (FIG. 23 B).

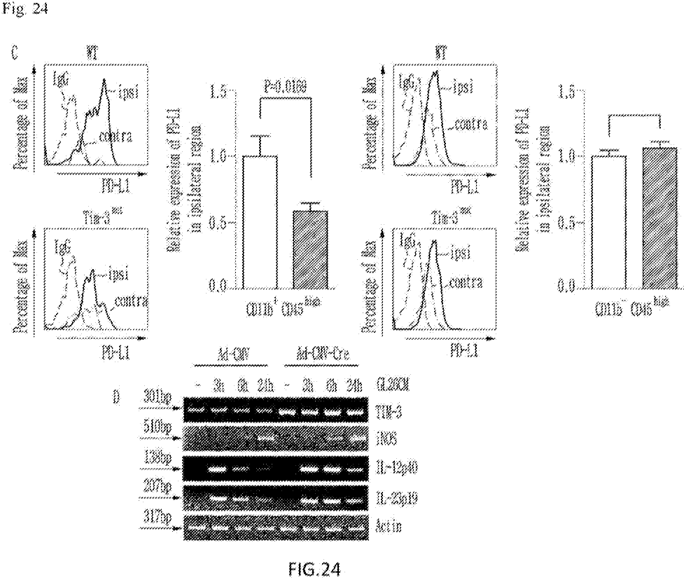

To further confirm the expression of PD-L1 in Tim-3mut mice, FACS analysis was used to examine PD-L1 expression levels in GL26 tumor-bearing mice in CD11b⁺CD45^{mid} microglial cells and CD11b⁺CD45^{high} macrophages. PD-L1 expression was significantly reduced in microglial cells and macrophages from GL26 glioma-containing regions of Tim-3mut mice compared to WT mice (FIG. 7E, FIG. 24C, WT = 5, Tim-3mut = 7). The graph showed the relative levels of PD-L1 from three independent experiments.

Additionally, primary glial was used from flox-stop-flox TIM-3 (FSF-TIM-3) mice, in which TIM-3 expression was driven in a Cre-dependent manner. Primary glial cells cultured from FSF-TIM-3 mice were infected with either CMV-adenovirus (Ad-CMV) or Cre recombinant adenovirus (Ad-CMV-Cre) and treated with GL26CM for the indicated times. RT-PCR analysis was performed using the indicated primers. Results represent at least 3 independent experiments. According to RT-PCR analysis or quantitative real-time PCR, it was shown that the transcriptional levels of the immune-related genes described above were more strongly induced in TIM-3high glial cells infected with Ad-CMV-Cre compared to control cells infected with Ad-CMV (FIG. 8a F, G and FIG. 24 D). These results suggested that defects in TIM-3 signaling or high-level expression of TIM-3 affected the expression of several immune-related genes, including PD-L1, including immune and inflammatory signaling.

### Example 4 Effect of Brain Tumor CM or Pam3CSK4 on TIM-3 and TLR2 in Glial

TLR2 recognizes exogenous and endogenous pathogens and regulates numerous inflammation-related genes, including iNOS and PD-L1. Considering that the induction of iNOS and PD-L1 by GL26CM or Pam3CSK4 (typically a TLR2 ligand) was significantly reduced in Tim-3mut glia, to determine whether TLR2 is associated with TIM-3-mediated signaling in glia, we investigated the effect of GL26CM or Pam3CSK4 on TLR2 levels, along with expression levels of TLR1, -2 and -6 in WT and Tim-3mut glia. The graph showed the relative levels of TLR2 in at least 3 independent experiments.

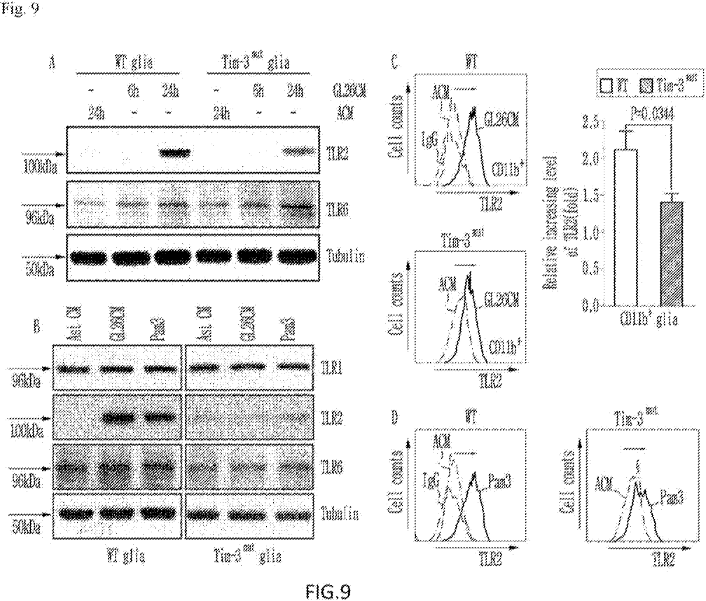

As a result, Western blot analysis showed that the level of TLR2 was significantly increased by GL26CM or Pam3CSK4, which level was significantly lower in Tim-3mut glia (FIG. 9 A, B). However, there was no significant difference in the expression of TLR1 or TLR6. FACS analysis confirmed that the induction of TLR2 by GL26CM or Pam3CSK4 (100 ng/ml) was less in CD1 1b+ glial of Tim-3mut mice compared to WT mice (FIG. 9 C, D).

Additionally, to determine whether TLR2 and TIM-3 had a reciprocal effect on glia in the brain tumor microenvironment, we compared cell surface expression levels of TIM-3 on primary glial cells in WT, TLR2-knockout (KO), and TLR4-KO mice. Primary glial glia from WT, TLR2-KO and TLR4-KO mice were treated with GL26CM or 100 ng/ml Pam3CSK4 for 24 h, and the expression levels of TIM-3, PD-L1 and TLR2 were measured by flow cytometry. Data represent at least 3 independent experiments.

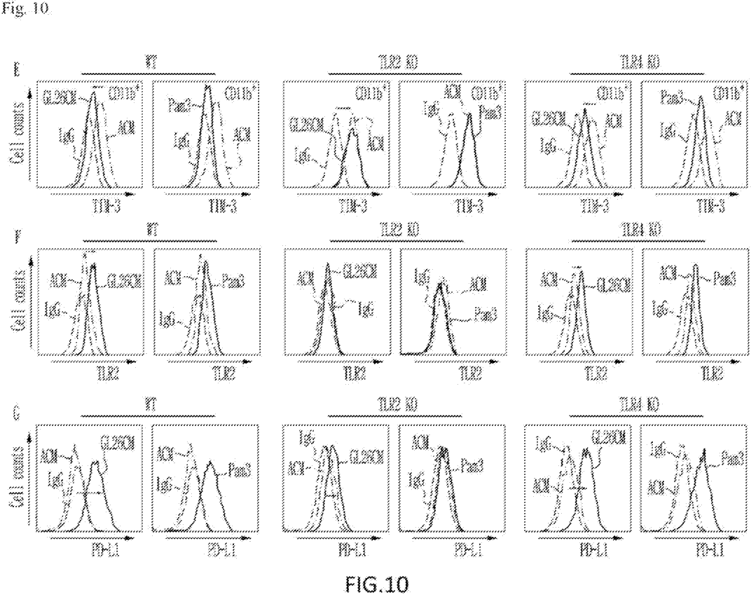

As a result, the level of TIM-3 in WT or TLR4-deficient glial was lower and the level of TLR2 was higher when treated with GL26CM or Pam3CSK4 compared to that treated with normal astrocyte CM (FIG. 10 E, F). On the other hand, no decrease in TIM-3 or increase in TLR2 was observed in TLR2-deficient glia treated with GL26CM or Pam3CSK4. Under the same conditions, either GL26CM or Pam3CSK4-induced increases in PD-L1 expression were reduced in TLR2-deficient glial compared to WT and TLR4-deficient glial (FIG. 10 G).

### Example 5 Effect of Glial TIM-3 on the Interaction of Glial and CD8+ T Cells

Glial cells can respond to pathological stimuli and activate T cells, including CD8+ T cells. To define the function of glial TIM-3 in the brain tumor microenvironment, we determined whether glial TIM-3 dysfunction could affect the interaction between glial and CD8+ T cells using an in vitro co-culture system. Primary glia of Tim-3mut or WT mice were cultured, respectively, and CD8+ T cells were isolated from lymph nodes of WT mice. Primary glial cells were plated in 96-well plates (3 × 10⁴ cells/ well) and pretreated for 24 h in the presence or absence of GL26CM. After MACS isolation and stimulation by CD3 for 1 h, either alone or in combination with CD8+ T cells (T cells: Glia = 1: 0.1), cultured for 48 hours, 100 µl of conditioned medium was collected and the level of IFN-γ secreted into the medium was analyzed with an ELISA kit for mouse IFN-γ (eBioscience) according to the manufacturer’s instructions. Results represented at least 3 independent experiments. Relative expression levels were expressed as mean fold change (± SD).

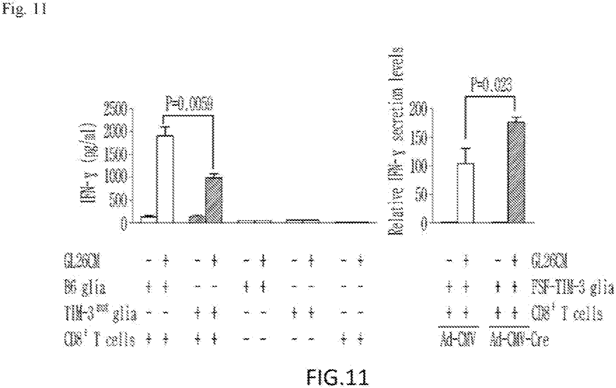

IFN-γ secretion was significantly increased in the presence of GL26CM when primary WT glial and CD8+ T cells were co-cultured. These enhancement was significantly reduced in co-cultures containing Tim-3mut glial and CD8+ T cells (FIG. 11 A). IFN-γ secretion was not noticeable only in primary glial or CD8+ T cells.

Additionally, we infected primary glial cells into FSF-TIM-3 mice with Ad-CMV or Ad-CMV-Cre, and pretreated the cells with ACM or GL26CM for 24 h. After that, the level of IFN-γ was analyzed by ELISA by incubation with CD8+ T cells for 48 hours.

As a result, IFN-γ levels were significantly higher in TIM-3high glial cells infected with Ad-CMV-Cre compared to control cells infected with Ad-CMV (FIG. 11 B). This meant that glial TIM-3 could affect the secretion of IFN-γ by CD8+ T cells in the presence of brain tumors.

### Example 6 Effect of Hypoxia on TIM-3 Expression in the Brain

Glial TIM-3 expression is regulated by hypoxia in a HIF-1-dependent manner, and hypoxia is a hallmark of the tumor microenvironment. Accordingly, the present inventors determined whether the level of neural tumor TIM-3 was affected by hypoxia caused by brain tumor CM.

Specifically, primary glial cells were cultured and incubated with GL26CM or ACM control for 24 hours under 20% or 1% O₂ conditions for the indicated times. RT-PCR analysis was performed using two sets of TIM-3 primers designed from different sequences. The graph showed the relative levels of TIM-3 obtained from at least three independent experiments.

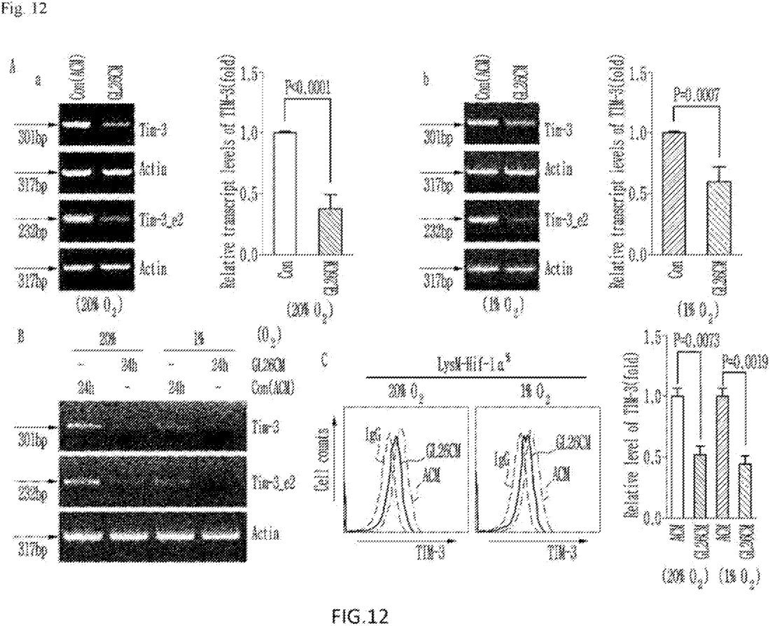

As a result, similar to the results obtained under 20% O₂, TIM-3 transcript levels were reduced by GL26CM under 1% O₂ (FIG. 12A).

Additionally, we investigated whether TIM-3 expression was altered by GL26CM in HIM-1α-deficient primary glia. Primary glial glia from LysM-Hif-1α ^{-/-}mice were transfected with Cre-expressing adenovirus for depletion of HIF-1 α and then incubated with GL26CM or ACM in normoxic or hypoxic environment for 24 h. TIM-3 transcript (B) and cell surface (C) levels of TIM-3 were confirmed by RT-PCR and FACS analysis.

As a result, RT-PCR and FACS analysis showed that TIM-3 expression was reduced by GL26CM in LysM-Hif-1α^{-/-} glia under 20% O₂ and 1% O₂ (FIG. 12 B, C).

Additionally, CD8+ T cells isolated from the spleen of C57BL/6 mice were treated with 5 µg/ml anti-CD3, 2 µg/ml anti-CD28, and 20 ng/ml IL-2 under normoxia and hypoxia for 12 hours. After incubation for 24 h under 20% O₂ and 1% O₂ with ACM or GL26CM, the level of TIM-3 was confirmed by flow cytometry.

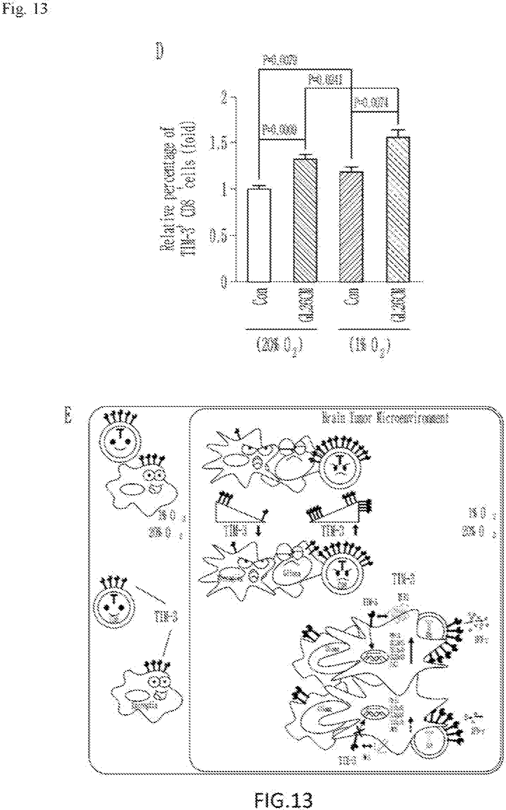

As a result, TIM-3 levels of CD8+ T cells were slightly increased by GL26CM in normoxic and hypoxic environments (FIG. 13D). The increase in TIM-3 by GL26CM in CD8+ T cells was similar at 20% O₂ and 1% O₂, but TIM-3 levels were higher in 1% O₂ than in 20% O₂. These results suggested that tumor-dependent alterations of TIM-3 expression could occur irrespective of oxygen levels in the brain tumor microenvironment.

1. A method of providing information for diagnosing cancer or predicting prognosis of cancer, comprising the step of measuring the expression of TIM-3 in CD11b+ cells and judging it as cancer or predicting that cancer has recurred when the level is lower than that of a normal control group.

2. The method of claim 1, wherein the CD11b+ cells are glia cells, myeloid cells, or peripheral blood mononuclear cells (PBMCs).

3. The method of claim 2, wherein the glial cells are at least one selected from the group consisting of radioactive glia cells, astrocytes, oligodendrocytes, oligodendrocytes progenitor cells and microglia.

4. The method of claim 2, wherein the myeloid cells are any one or more selected from the group consisting of leukocytes, mast cells, monocytes, macrophages, and dendritic cells.

5. The method of claim 1, wherein the cancer is a brain tumor.

6. A composition for diagnosing cancer or predicting prognosis of cancer comprising an agent for measuring the expression level of TIM-3 in CD11b+ cells.

7. A kit for diagnosing cancer or predicting prognosis of cancer comprising the composition of claim 6.
